(19) United States
(12) Patent Application Publication
(10) Pub. No.: US 2023/0357703 A1
(43) Pub. Date: Nov. 9, 2023

(54) METHODS AND APPARATUS FOR BIOPROCESS MONITORING

(71) Applicant: Global Life Sciences Solutions USA LLC, Marlborough, MA (US)

(72) Inventors: Aravind Kumar Viswanathan, Bengaluru (IN); Jaisimha Seshadri, Singapore (SG); Christopher Sandusky, Marlborough, MA (US); Suthish Kumar S, Bengaluru (IN); Shanmuganathan Ca, Bengaluru (IN); Sasikumar Arumugam, Bengaluru (IN)

(21) Appl. No.: 18/247,728

(22) PCT Filed: Sep. 22, 2021
(86) PCT No.: PCT/EP2021/076015

§ 371 Date: Apr. 3, 2023

(30) Foreign Application Priority Data
Oct. 3, 2020 (IN) 202011043053

(51) Int. Cl. C12M 1/36; G16B 40/00

(52) U.S. Cl. CPC C12M 41/48; G16B 40/00

(57) ABSTRACT

Methods and apparatus for bioprocess monitoring are disclosed. An example apparatus includes a controller to monitor a first bioprocess instrument, a data logger to collect data for the first bioprocess instrument, the collected data including data collected while the first bioprocess instrument is transferred from a first location to a second location, a configurator to configure the first bioprocess instrument to operate in a first mode at the first location and in a second mode at the second location, the first or second mode determined based on a type of processing at the first or second location, and a user interface to display the collected data to a user, the collected data including real-time bioprocess monitoring data, the controller to adjust a setting of the first bioprocess instrument based on the monitoring data, the monitoring data used to maintain controlled environmental conditions within a vessel of the instrument.

### RELATED APPLICATIONS

This application claims priority to Patent Cooperation Treaty (PCT) Application Number PCT/EP2021/076015, filed on Sep. 22, 2021, which claims priority to Indian Provisional Application No. 202011043053, filed Oct. 3, 2020. PCT Application Number PCT/EP2021/076015 and Indian Provisional Application No. 202011043053 are incorporated by reference herein in their entireties.

### FIELD OF THE DISCLOSURE

This disclosure relates generally to bioprocess systems and, more particularly, to methods and apparatus for bioprocess monitoring.

### BACKGROUND

Bioprocesses are used to produce medically and industrially critical products (e.g., therapeutics, biofuels, etc.) using biomanufacturing through optimization of natural and/or artificial biological systems to allow for large-scale production. Instruments for bioprocess control and analysis are used for maintaining optimal environmental conditions by monitoring and controlling operational variables (e.g., flow rate, temperature, pH, pressure, agitator shaft power, rate of stirring, etc.). As such, physical, chemical, and biological parameters must be kept constant or maintained at optimal levels to prevent any deviations from a set range. Such monitoring is present throughout the bioprocess, including upstream processing, during which biomass can be produced in large scales, as well as downstream processing, during which extraction/separation from the biomass and purification steps are used to yield the final product.

### BRIEF DESCRIPTION

Certain examples provide methods and apparatus for bioprocess monitoring.

Certain examples provide an apparatus including a controller to monitor a first bioprocess instrument, a data logger to collect data for the first bioprocess instrument, the collected data including data collected while the first bioprocess instrument is transferred from a first location to a second location. The example apparatus includes a configurator to configure the first bioprocess instrument to operate in a first mode at the first location and in a second mode at the second location, the first or second mode determined based on a type of processing at the first or second location. The example apparatus also includes a user interface to display the collected data to a user, the collected data including real-time bioprocess monitoring data, the controller to adjust a setting of the first bioprocess instrument based on the monitoring data, the monitoring data used to maintain controlled environmental conditions within a vessel of the instrument during ongoing processing when the instrument is in transit from the first location to the second location.

Certain examples provide a computer-implemented method, the method including operating a first bioprocess instrument in a first mode, the first mode based on at least one of a first location or a first bioprocess task to be performed by the first instrument and monitoring the first bioprocess instrument during a transfer of the first bioprocess instrument from the first location to a second location, the monitoring including collection of real-time bioprocess monitoring data. The example method also includes configuring the first bioprocess instrument to operate in a second mode based on at least one of the monitoring data, the second location, or a second bioprocess task, and operating the first bioprocess instrument in the second mode in the second location, a setting of the instrument modified based on the monitoring data.

Certain examples provide at least one computer-readable storage medium including instructions that, when executed, cause a machine to at least operate a first bioprocess instrument in a first mode, the first mode based on at least one of a first location or a first bioprocess task to be performed by the first instrument and monitor the first bioprocess instrument during a transfer of the first bioprocess instrument from the first location to a second location, the monitoring including collection of real-time bioprocess monitoring data. The example instructions further cause the machine to configure the first bioprocess instrument to operate in a second mode based on at least one of the monitoring data, the second location, or a second bioprocess task, and operate the first bioprocess instrument in the second mode in the second location, a setting of the instrument modified based on the monitoring data.

### BRIEF DESCRIPTION OF THE DRAWINGS

FIG. 1 is a block diagram illustrating an example known environment for bioprocess instrument monitoring.

FIG. 2 is a block diagram illustrating an example environment for wireless bioprocess instrument monitoring in accordance with the teachings of this disclosure.

FIG. 3 is a block diagram illustrating an example environment for wireless bioprocess instrument monitoring and data logging in accordance with the teachings of this disclosure, including an example user interface and an example bioprocess unit tracker.

FIG. 4 is a block diagram of an example implementation of the bioprocess unit tracker of FIG. 3 to facilitate bioprocess instrument monitoring.

FIG. 5 is a flowchart representative of example machine-readable instructions that may be executed to monitor a bioprocess instrument.

FIG. 6 is a flowchart representative of example machine-readable instructions that may be executed to collect and assess real-time bioprocess monitoring data.

FIG. 7A depicts an example data flow diagram for managing communication among an example controller, an example communication interface, and an example transmitter/receiver of a bioprocess instrument at a first location.

FIG. 7B depicts a data flow diagram for managing communication among an example controller, an example communication interface, and an example transmitter/receiver of a bioprocess instrument at a second and a third location.

FIG. 8 is a block diagram of an example processing platform structured to execute the example instructions of FIGS. 5-6 to implement the example controller of FIGS. 2 and 3.

FIG. 9 is a block diagram of an example processing platform structured to execute the example instructions of FIGS. 5-6 to implement the example bioprocess unit tracker of FIGS. 3 and 4.

The figures are not scale. Wherever possible, the same reference numbers will be used throughout the drawings and accompanying written description to refer to the same or like parts.

### DETAILED DESCRIPTION

In the following detailed description, reference is made to the accompanying drawings that form a part hereof, and in which is shown by way of illustration specific examples that may be practiced. These examples are described in sufficient detail to enable one skilled in the art to practice the subject matter, and it is to be understood that other examples may be utilized and that logical, mechanical, electrical and other changes may be made without departing from the scope of the subject matter of this disclosure. The following detailed description is, therefore, provided to describe an exemplary implementation and not to be taken as limiting on the scope of the subject matter described in this disclosure. Certain features from different aspects of the following description may be combined to form yet new aspects of the subject matter discussed below.

“Including” and “comprising” (and all forms and tenses thereof) are used herein to be open ended terms. Thus, whenever a claim employs any form of “include” or “comprise” (e.g., comprises, includes, comprising, including, having, etc.) as a preamble or within a claim recitation of any kind, it is to be understood that additional elements, terms, etc. may be present without falling outside the scope of the corresponding claim or recitation. As used herein, when the phrase “at least” is used as the transition term in, for example, a preamble of a claim, it is open-ended in the same manner as the term “comprising” and “including” are open ended. The term “and/or” when used, for example, in a form such as A, B, and/or C refers to any combination or subset of A, B, C such as (1) A alone, (2) B alone, (3) C alone, (4) A with B, (5) A with C, (6) B with C, and (7) A with B and with C. As used herein in the context of describing structures, components, items, objects and/or things, the phrase “at least one of A and B” is intended to refer to implementations including any of (1) at least one A, (2) at least one B, and (3) at least one A and at least one B. Similarly, as used herein in the context of describing structures, components, items, objects and/or things, the phrase “at least one of A or B” is intended to refer to implementations including any of (1) at least one A, (2) at least one B, and (3) at least one A and at least one B. As used herein in the context of describing the performance or execution of processes, instructions, actions, activities and/or steps, the phrase “at least one of A and B” is intended to refer to implementations including any of (1) at least one A, (2) at least one B, and (3) at least one A and at least one B. Similarly, as used herein in the context of describing the performance or execution of processes, instructions, actions, activities and/or steps, the phrase “at least one of A or B” is intended to refer to implementations including any of (1) at least one A, (2) at least one B, and (3) at least one A and at least one B.

As used herein, singular references (e.g., “a”, “an”, “first”, “second”, etc.) do not exclude a plurality. The term “a” or “an” entity, as used herein, refers to one or more of that entity. The terms “a” (or “an”), “one or more”, and “at least one” can be used interchangeably herein. Furthermore, although individually listed, a plurality of means, elements or method actions may be implemented by, e.g., a single unit or processor. Additionally, although individual features may be included in different examples or claims, these may possibly be combined, and the inclusion in different examples or claims does not imply that a combination of features is not feasible and/or advantageous.

Bioprocess monitoring requires real-time, continuous measurement of process variables to ensure the stability, efficiency, and reproducibility of the process to provide for a high-quality product. By measuring quality-related process variables that are necessary to maintain a narrow range of environmental conditions, consistent reproduction of the desired product can be achieved and documented. A variety of bioprocess instruments (also referred to herein as bioprocess units) are used during upstream processing (e.g., biomass expansion, media development and preparation, etc.) and downstream processing (e.g., product extraction and purification from the biomass, etc.), including bioreactors and mixers. For example, a bioreactor can be used to create a controlled environment for in vitro management of cells (e.g., cell proliferation, differentiation, etc.) during upstream processing. Bioreactors can include sensors directly interfacing, or used in conjunction with, the bioreactors to measure process variables, including oxygen and carbon dioxide concentration, biomass concentration, flow injection, and/or overall media composition. Other units used during upstream processing can include media storage tanks, media exchangers, and/or mixers. Downstream processes focus on optimizations to extract and maximize final product yields, including filtration, mixing, and purification based on chromatography. Any variability in upstream processing directly affects downstream processing, potentially impacting processing time and final product quality. As such, real-time and continuous monitoring is vital to a well-documented biomanufacturing process and permits timely intervention and mitigation of potential issues that can affect quality control.

Bioprocess units can be configured to meet specific process demands, allowing process scale-up and large-scale biomanufacturing (e.g., biopharmaceutical manufacturing). Such units can include a built-in computer with dedicated software that permits stand-alone operation or integration into a plant-wide control system. Connection of the units to a local control system can be used to achieve real-time unit monitoring. For example, network integration can permit user-based operation of the unit with remote logging of historical data. Full control of such units can be achieved using a digital communications system for manufacturing and process automation, such as a Process Field Bus (PROFIBUS) gateway (e.g., PI-960) connection to a local control system, EthernetIP, or any other type of wired network (e.g., Controller Area Network (CAN), RS-485, etc.). For example, devices on the system can connect to a network and permit communication between a control system and the field devices (e.g., bioprocess units). However, such units can be connected only to select ports within a specific wired network, with the addition of new units requiring physical device reconfiguration instead of facilitating discovery of the hardware component on the system via plug and play (PnP) computing (e.g., automatic detection and configuration of hardware). In some cases, connection of numerous units results in lengthy wiring (e.g., more than 200 meters) and introduces potential failure points as well as cumbersome changes during unit replacement and/or servicing.

Additionally, any movement of the unit from one location to another location can require disconnection of the unit from the network, thereby eliminating data monitoring and data logging during unit transit. For example, a mixer being moved from one room to another is disconnected from the wired network (e.g., PROFIBUS, EthernetIP, CAN, RS-485, etc.), with no real-time data monitoring of critical parameters (e.g., temperature, pH, conductivity, etc.), as well as no real-time agitation control (e.g., agitator shaft power, rate of stirring, etc.). As such, real-time, continuous monitoring of such parameters is not possible while the unit is in motion and not physically connected to the network from one location to another location. Limited process monitoring not only reduces process-related data collection, but also affects the quality and quantity of bioprocess product yields (e.g., product titer and yield), lot-to-lot consistencies, and overall productivity of the system. Accurate, real-time monitoring of physical, chemical, and/or biological parameters contributes to the production of high-quality products and improves overall bioprocess efficiency. Further limitations of using a wired network (e.g., PROFIBUS, EthernetIP, CAN, RS-485, etc.) may include slower device and Input/Output (I/O) communication (e.g., 128 Kbps) due to existing protocol limitations. Such limitations affect scanning time and result in reduced data exchange rates. Likewise, the total number of units used in plant floor operations may be limited (e.g., a total of 25 mixers) and dedicated to a specific location, remaining stationary and unused during the bioprocess rather than being mobile with flexible location assignments (e.g., during batch system synchronization of steps).

Methods and apparatus for bioprocess monitoring described herein permit improved bioprocess unit tracking and real-time, continuous data monitoring and data logging for a unit in transit between different locations on the plant floor. Methods and apparatus described herein further permit optimization and improved efficiency of the biomanufacturing process by allowing bioprocess units to be used in upstream processing and/or downstream processing at multiple stages and/or batch collection points, as opposed to remaining stationary as a dedicated unit at a single location, thereby increasing the unit's rate of utilization. Real-time monitoring and continuous data collection during unit transit ensures tracking of critical parameters (e.g., temperature, pH, conductivity, etc.) to ensure stable and consistent environmental conditions within the unit as necessitated by a specific bioprocess task and/or operation. Methods and apparatus disclosed herein permit replacement of a wired network (e.g., PROFIBUS, EthernetIP, CAN, RS-485, etc.) cabling with a wireless Internet Protocol (IP) address (e.g., wireless ethernet IP), while a primary wireless transmit/receive (TX/RX) interface exchange replaces wired network repeater cabinets (e.g., used to regenerate a transmission signal and resend the signal to eliminate faults in the line).

Furthermore, methods and apparatus disclosed herein permit real-time auto-configuration detection between a primary wireless hub and wireless transmitter(s)/receiver(s) in communication with one or more bioprocess units distributed throughout a manufacturing plant. For example, methods and apparatus disclosed herein permit plug and play (PnP) computing, allow for automatic handshaking (e.g., establishing and verifying a connection), support alarm notification and diagnostics for data exchanges, and improve security by re-using IP addresses via Network Address Translation (NAT). As such, bioprocess units can be wirelessly monitored in transit, improving their mobility throughout a plant by allowing the same unit to be moved to various areas, as determined by the timing and location of the downstream/upstream processing, thereby maximizing or otherwise improving throughput, efficiency, flexibility, and configurability of the biomanufacturing process.

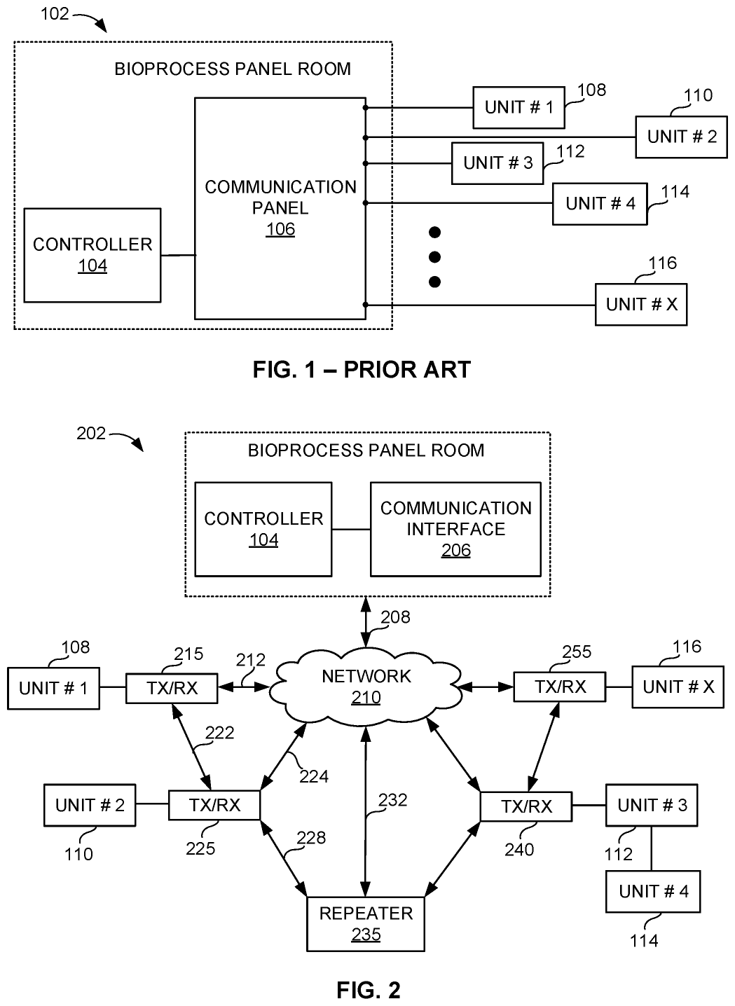

FIG. 1 is a block diagram illustrating an example conventional environment 102 for bioprocess instrument monitoring. The known environment 102 includes a bioprocess panel room with an example controller 104, an example communication panel 106, an example first unit 108 (e.g., unit #1), an example second unit 110 (e.g., unit #2), an example third unit 112 (e.g., unit #3), an example fourth unit 114 (e.g., unit #4), and an example fifth unit 116 (e.g., unit #x). The controller 104 controls the bioprocess units 108, 110, 112, 114, 116 via the communication panel 106, allowing data logging and monitoring of any of the units 108, 110, 112, 114, 116 connected to a wired network (e.g., PROFIBUS, Controller Area Network (CAN), RS-485, etc.). For example, the controller 104 can monitor the bioprocess units 108, 110, 112, 114, 116 to determine if any critical parameters (e.g., temperature, pH, pressure, oxygen, carbon dioxide, etc.) are fluctuating and/or outside a designated range required to maintain a controlled environment during upstream and/or downstream processing. In some examples, the controller 104 is a programmable logic controller (PLC) designed specifically for the control of manufacturing processes (e.g., biomanufacturing). In some examples, the PLC can include external Input/Output (I/O) modules attached to a fieldbus or a computer network that plugs into the controller.

The communication panel 106 connects the units 108, 110, 112, 114, 116 to the local control system (e.g., the controller 104). The communication panel 106 can be any type of wired network (e.g., PROFIBUS, EthernetIP, CAN, RS-485, etc.) or gateway (e.g., PROFIBUS PI-960) used for digital communications in manufacturing and process automation. The addition of new or replacement units to the local control system via the communication panel 106 requires physical device reconfiguration to allow for detection of the hardware on the system network. In some examples, connection of numerous units results in lengthy wiring (e.g., over a few hundred meters) and can require cumbersome changes during unit replacement and/or servicing.

The bioprocess units 108, 110, 112, 114, 116 can be any type of unit and/or instrument used during biomanufacturing, from initial biomass expansion and media preparation to final product collection and purification. For example, one or more of the units 108, 110, 112, 114, 116 can be a bioreactor (e.g., a stirred tank bioreactor, an airlift bioreactor, etc.), a mixer (e.g., a jacketed mixer, a single wall mixer, etc.), a fermenter, or any other type of equipment using during downstream/upstream processing (e.g., scales, pumps, single-use filtration systems, etc.). For example, a bioreactor can be used for expansion (e.g., growth of CHO cells, bacteria, yeast, etc.) to permit biological reactions under controlled conditions for a variety of purposes, including the production of pharmaceuticals, vaccines, antibodies, and biofuel. Such bioreactors can be used in any domain of industrial biotechnology requiring large scale production, providing the necessary biological, biochemical, and biomechanical conditions for synthesis of desired products. Mixers can be used during biomanufacturing for buffer and media preparations or other mixing needs (e.g., blending, stirring, suspending, dissolving, etc.). Mixers include single-use mixing platforms with disposable bag sizes ranging from 20-2,000 liters, the disposable bags reducing risks of contamination, as well as reducing the need for additional cleaning and sterilization. Additional bioprocess equipment can include filtration and chromatography systems, used in the purification of biopharmaceutical products.

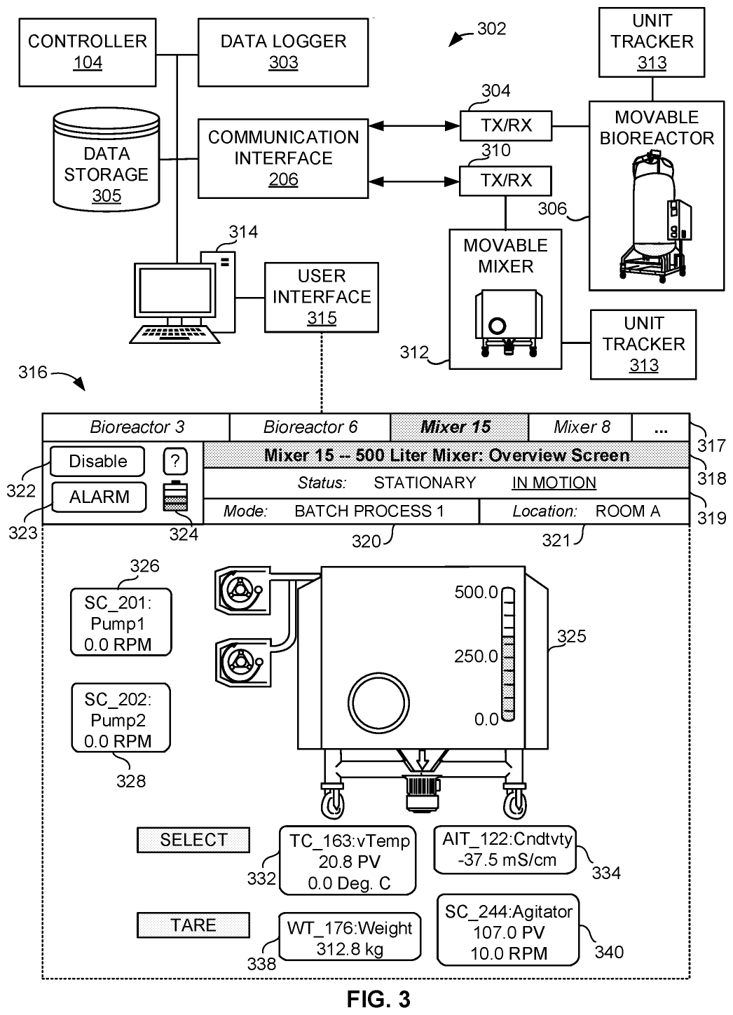

FIG. 2 is a block diagram illustrating an example environment 202 for wireless bioprocess instrument monitoring in accordance with the teachings of this disclosure. The example environment 202 includes a bioprocess panel room with a communication interface 206 and the controller 104. The controller 104 is in communication with bioprocess units 108, 110, 112, 114, 116 via a wireless network represented by example network 210. The network 210 can be implemented using any suitable wireless network(s) including, for example, one or more data buses, one or more Local Area Networks (LANs), one or more wireless LANs, one or more cellular networks, the Internet, etc. As used herein, the phrase “in communication,” including variances thereof, encompasses direct communication and/or indirect communication through one or more intermediary components and does not require direct physical (e.g., wired) communication and/or constant communication, but rather additionally includes selective communication at periodic or aperiodic intervals, as well as one-time events. The communication interface 206 selectively communicates with the bioprocess units 108, 110, 112, 114, 116 via example wireless transmitter/receivers (TX/RX) 215, 225, 240, 255, each of which are connected to one or more bioprocess units (e.g., units 108, 110, 112, 114, 116). The environment 202 also includes an example repeater 235 to regenerate a transmission signal and resend it, thereby amplifying the wireless signal in any potential “dead zone” areas with reduced coverage.

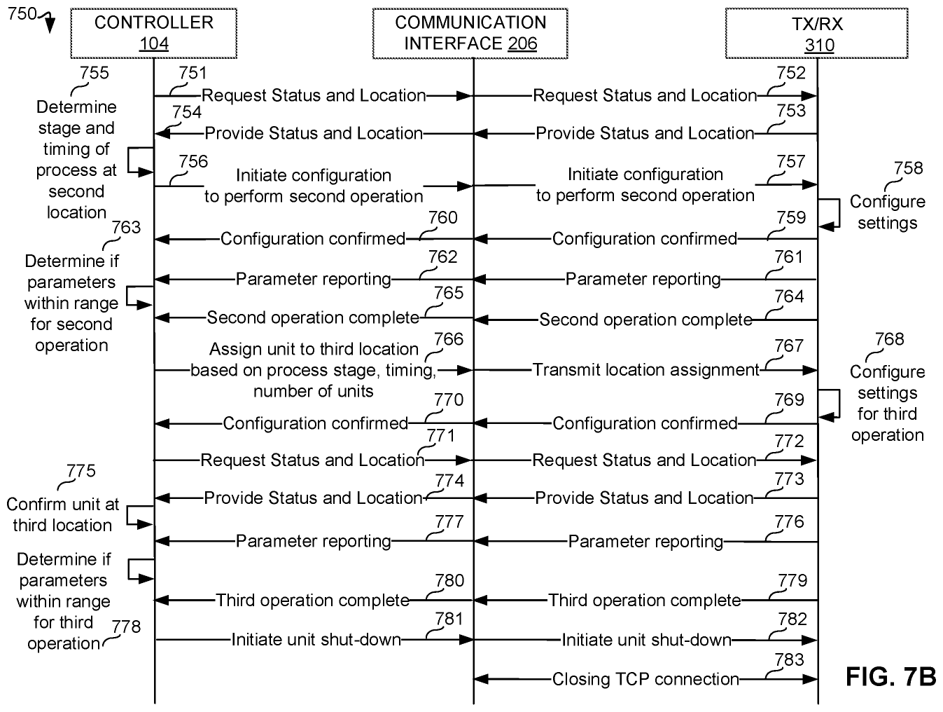

The communication interface 206 replaces the communication panel 106 of FIG. 1. The communication interface 206 can communicate with the bioprocess units 108, 110, 112, 114, 116 via wired and/or wireless-based Ethernet, providing a high-power industrial hotspot for exchange of high speed data (e.g., data exchange rates of up to 54 Megabytes per second (Mbps) and signal range support of up to 8 kilometers). In some examples, the communication interface 206 permits real-time autoconfiguration of units 108, 110, 112, 114, 116 via plug and play (PnP) computing, instead of requiring physical device reconfiguration when new units are connected and/or added to the network. As such, when the unit 108, 110, 112, 114, 116 is in motion or stationary, the unit can register specific hardware and/or software configurations with a host without requiring additional configuration and/or re-configuration (e.g., when switching modes, being powered on, etc.). In some examples, the communication interface 206 permits identification of a unit's (e.g., unit 108, 110, 112, 114, 116) location. Likewise, the use of a wireless environment 202 of FIG. 2 permits automatic handshaking between the communication interface 206 and one or more of the bioprocess units 108, 110, 112, 114, 116. For example, automatic handshaking allows for the setting of parameters between the communication interface 206 and one or more units 108, 110, 112, 114, 116 before normal communication begins (e.g., exchanging of protocol information, verifying the quality or speed of the connection, verifying any authority required to complete the connection, controlling data transmission, etc.). Additionally, handshaking can be used to authenticate the bioprocess unit(s) 108, 110, 112, 114, 116 and establish an encryption algorithm to be used for secure data transfer. For example, alarm notification(s) (e.g., critical alarms, notification of unit disconnection, etc.) and data exchanges used for diagnostics and data logging can occur via example communication(s) 212, 224 between the communication interface (e.g., via the network 210) and the wireless transmitter/receivers (TX/RX) 215, 225, respectively, as described in more detail in connection with data flow diagrams of FIGS. 7A-7B.

The wireless TX/RX 215, 225, 240, 255 can be connected to one or more of the units 108, 110, 112, 114, 116, as shown in the example of FIG. 2, allowing the controller 104 (e.g., a programmable logic controller) to transmit information and/or receive information from the units 108, 110, 112, 114, 116 via the communication interface 206 and/or the network 210. For example, a unit 108, 110, 112, 114, 116 can transmit information such as status (e.g., online, offline, operational, etc.), processing mode (e.g., upstream processing, downstream processing, batch number, etc.), location (room, floor, facility, etc.), battery usage (stationary and connected, moving and in battery mode, etc.), and/or any other information requested by the controller 104 and/or a user via the receiver (RX) (e.g., collected process data, critical parameter readings, etc.). In some examples, the wireless TX/RX 215, 225, 240, 255 can communicate information among multiple units (e.g., to determine which unit is available for a specific upstream/downstream processing task based on the total unit number, status, and/or availability, etc.). For example, unit 108 (e.g., unit #1) can receive and/or transmit information to unit 110 (e.g., unit #2) using the wireless TX/RX 215, 225, respectively, via example data transmission 222. In some examples, the repeater 235 can be used to amplify the wireless signal based on a network-initiated request 232 to ensure maximum signal strength for efficient data transfer. For example, the repeater 235 can amplify a signal received from the network 210 via example data transmission 228 to the wireless TX/RX 225 of unit 110 (e.g., unit #2). In some examples, a wireless TX/RX (e.g., wireless TX/RX 240) can service multiple units at once (e.g., units 112, 114). In some examples, unit 112 (e.g., unit #3) can be a production bioreactor and unit 114 (e.g., unit #4) can be an integrated scale or an integrated pump connected to the bioreactor, allowing the wireless TX/RX 240 to receive data from, and/or transmit data to, one or more of the units 112, 114 simultaneously.

As such, when any one or more of the units 108, 110, 112, 114, 116 are in motion (e.g., being moved from one location to another location), the example environment 202 of FIG. 2 permits ongoing, real-time tracking of the units 108, 110, 112, 114, 116 (e.g., critical parameter monitoring, process data collection, etc.), such that the units 108, 110, 112, 114, 116 are not limited to a designated area due to protocol limitations (e.g., as shown and described in connection with FIG. 1). For example, the wireless environment 202 increases scanning time and improved data exchange via faster device and I/O communication (e.g., 10× increase in data exchange speeds compared to using the PROFIBUS panel-based system of FIG. 1). Likewise, one or more new unit(s) #X (e.g., unit 116) can be added to the network via PnP-based computing without requiring additional cable installation, physical connection, and/or configuration to access the controller network (e.g., network 210), permitting faster product installation and supporting a larger number of units. The wireless environment 202 can also utilize Network Address Translation (NAT) to permit the connection of unit(s) 108, 110, 112, 114, 116 to a primary wireless hub without frequent changes to a unit's IP address (e.g., during unit re-location). The wireless environment 202 of FIG. 2 further facilitates usability and maintenance of bioprocess units via wireless and/or wired monitoring systems (e.g., tablet-based personal computers (PCs), industrial pendant PCs, mobile workstations, etc.).

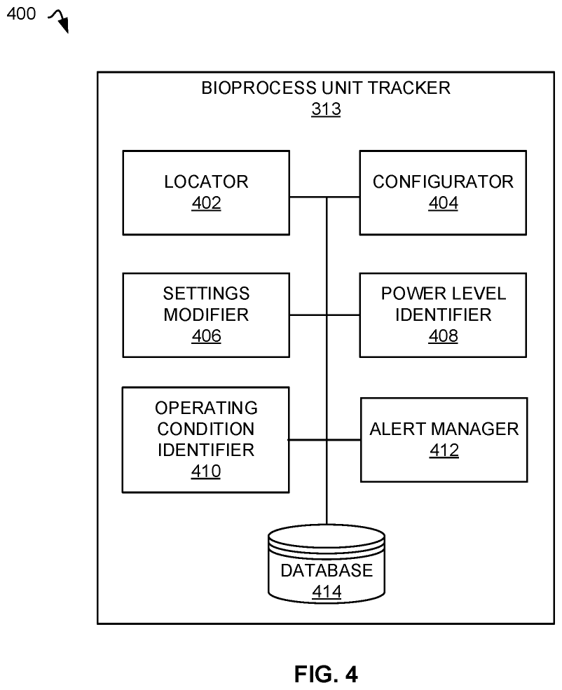

FIG. 3 is a block diagram illustrating an example environment 302 for wireless bioprocess instrument monitoring and data logging in accordance with the teachings of this disclosure. The environment 302 includes the controller 104, the communication interface 206, an example data logger 303, an example data storage 305, an example first wireless TX/RX 304, an movable bioreactor 306, an example second wireless TX/RX 310, an example movable mixer 312, an example bioprocess unit tracker 313, an example workstation 314, and an example user interface 315. The data logger 303 logs data relevant to the biomanufacturing process, including critical parameter monitoring (e.g., temperature, pH, conductivity, etc.) and/or other key indicators for evaluating a bioprocess unit's ability to maintain a controlled environment and ensuring sample quality and regulatory compliance. For example, the data logger 303 can receive data via the wireless TX/RX 304, 310 connected to the bioprocess units 306, 312 when the units are stationary or in motion (e.g., a bioprocess instrument is transferred from a first location to a second location). In some examples, the data logger 303 logs data received from on-line, real-time sensors and/or other automatic data acquisition systems associated with the bioprocess units.

The data storage 305 stores any logged data and/or any other information received from the bioprocess units 306, 312 during operation. In some examples, the data storage 305 includes data related to bioprocess unit 306, 312 location, status, mode, re-location, and/or other information related to bioprocess unit 306, 312 usage (e.g., maintenance, configuration, battery status, upstream/downstream processing tasks, etc.). The data storage 305 can be implemented by any storage device and/or storage disc for storing data such as, for example, flash memory, magnetic media, optical media, web-based storage, private cloud storage, etc. Furthermore, the data stored in the data storage 305 can be in any data format such as, for example, binary data, comma delimited data, tab delimited data, structured query language (SQL) structures, etc. While in the illustrated example the data storage 305 is illustrated as a single database, the data storage 305 can be implemented by any number and/or type(s) of databases. In some examples, the data storage 305 stores metadata, logged data, and time information as it is broadcast, to allow the data logger 303 to be used for applications developed to interpret, search, and report on the data.

In the example of FIG. 3, the controller 104 (e.g., a programmable logic controller) is in communication with the first and second wireless TX/RX 304, 310 via the communication interface 206. The first TX/RX 304 is connected to the movable bioreactor 306 (e.g., a production bioreactor), while the second TX/RX 310 is connected to the movable mixer 312 (e.g., a jacketed mixer). The bioreactor 306 can be used for expansion (e.g., growth of cells, bacteria, yeast, etc.) to permit biological reactions under controlled conditions. The mixer 312 can be used for buffer preparation, media preparation, harvest, clarification, and/or purification for intermittent storage. The mixer 312 can be based on any type of mixing technology, including motor-driven impellers, levitated magnetic stirrers, bellows systems with perforated plates, and/or magnetically-driven stir bars. However, any other type of bioprocess instrument can be used in the environment 302 of FIG. 3, either as a stand-alone bioprocess unit or as an integrated unit, including a fermenter, a pump, or a scale. The first TX/RX 304 provides monitoring data related to the bioreactor 306, allowing the controller 104 to evaluate whether the bioreactor is maintaining a proper controlled environment for biomass expansion (e.g., temperature, pH, oxygen, carbon dioxide, etc.). The controller 104 evaluates the critical parameters (e.g., logged via the data logger 303) to determine whether they are within an acceptable range and/or require adjustment. In some examples, the bioreactor 306 is moved from one area of the plant floor to another area, depending on processing needs. In some examples, the data logger 303 records a mode of the bioreactor 306 operation (e.g., batch, fed-batch, continuous, perfusion processing, etc.) while the bioreactor 306 is stationary or in motion. The data logger 303 continues to receive and store information (e.g., in the data storage 305) relevant to process parameters while the bioreactor 306 is stationary or in motion. In some examples, the controller 104 monitors and/or optimizes various conditions and/or parameters that affect the results of the bioreactor-based scale-up (e.g., gas distribution, mixing time, heat-transfer rate, mass-transfer coefficients, volumetric power input, etc.). Additionally, the data logger 303 can log real-time pH readings and/or osmolarity readings (e.g., using near-infrared spectroscopy, optical sensors, etc.), while biomass production can be monitored using radio-frequency impedance methods.

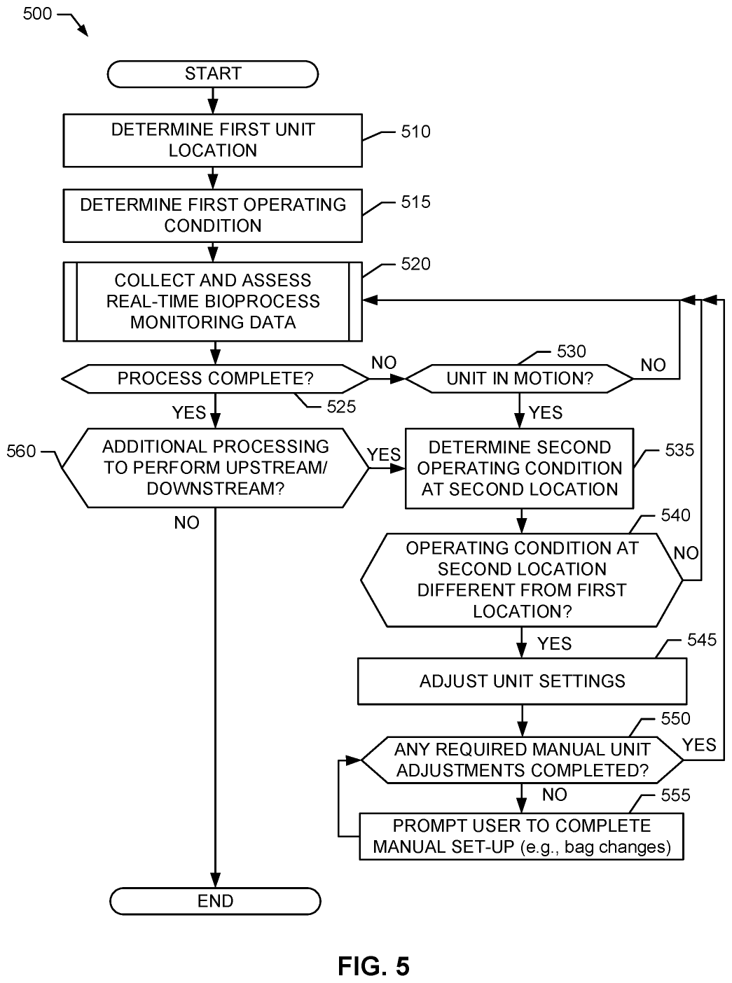

The second TX/RX 310 provides monitoring data related to the mixer 312, allowing the controller 104 to perform the same level of monitoring of process control variables (e.g., pH, temperature, etc.) for the mixer 312 as with the bioreactor 306. In the example of FIG. 3, the mixer 312 is in communication with a bioprocess unit tracker 313. The unit tracker 313 facilitates the identification, collection, and/or transfer of process-relevant data to the controller 104 and/or the data logger 303. As described in more detail in connection with FIG. 4, the unit tracker 313 determines unit location, configures/reconfigures the unit, identifies the unit's operating condition (e.g., mode, status, etc.), generates and/or manages alerts, modifies settings, and/or identifies the unit's power level. As such, the unit tracker 313 functions as a dedicated, local unit manager for purposes of transmitting information requested by the controller 104 and/or processing tasks to be performed by the unit as initiated by the controller 104. While in the example of FIG. 3 the unit tracker 313 is shown to be in communication with the mixer 312 and/or the bioreactor 306, the unit tracker 313 can be a software-based program that is dedicated to any one or more of the units 108, 110, 112, 114, 116 of FIG. 2.

The workstation 314 permits a user to operate and/or monitor the bioprocess units, including the units 306, 312 of FIG. 3. The workstation 314 is communicatively coupled to the controller 104, the communication interface 206, the data logger 303, and/or the data storage 305 via a bus or local area network (LAN) (e.g., an Area Control Network (ACN)). The LAN can be implemented using any desired communication medium and protocol. For example, the LAN can be based on a hardware or wireless Ethernet communication protocol. However, any other suitable wired or wireless communication medium and protocol could be used. The workstation 314 can be configured to perform operations associated with one or more information technology applications, user-interactive applications (e.g., via the user interface 315), and/or communication applications. For example, the workstation 314 can be configured to perform operations associated with process control-related applications and communication applications that enable the workstation 314 and the controller 104 to communicate with other devices or systems using any desired communication media (e.g., wireless, hardwired, etc.) and protocols (e.g., HTTP, SOAP, etc.).

An example user interface display 316 can be generated by the user interface 315 of the workstation 314. The user interface display 316 includes example screen navigation tab 317, example unit identification tab 318, example motion status identification tab 319, example mode identification tab 320, example location identification tab 321, an example disable button 322, an example alarm display 323, an example battery indicator 324, and an example diagram 325 of the unit being viewed. The screen navigation tab 317 of the user interface display 316 allows a user to navigate from one screen to another (e.g., viewing information for Bioreactor 3, Bioreactor 6, Mixer 15, Mixer 8, etc.) to view information related to the unit being selected for viewing. In the example of FIG. 3, one of the mixing units is selected by a user, thereby resulting in the unit identification tab 318 displaying “Mixer 15” with additional unit characteristics (e.g., total unit capacity, etc.). The motion status identification tab 319 displays to the user whether the selected unit (e.g., Mixer 15) is stationary or in motion. In the example of FIG. 3, the motion status identification tab 319 indicates that the selected unit is in motion (e.g., being moved from a first location to a second location). As shown in the example of FIG. 3, the user interface 315 continues to display real-time, continuous unit monitoring whether the unit is in motion or stationary. The mode identification tab 320 displays a processing mode of the selected unit. For example, the processing mode for a bioreactor can be identified as batch, fed-batch, continuous, and/or perfusion processing. The location identification tab 321 displays the real-time location of the unit in the biomanufacturing facility, including a specific facility, floor, room, etc. The location identification tab 321 is updated in real-time as the unit is moved from a first location to a second location. In some examples, an interactive display and/or indicator of unit location on a map of the biomanufacturing facility can be show to the user to better orient them to the location and/or movement of the unit. The disable button 322 permits a user to disable the unit and/or abort an operation in situations that warrant such termination (e.g., an alarm indicating that collected data deviates from a tolerance limit for a nominal value). The alarm display 323 can indicate to the user any deviations of the unit from normal settings and/or operations. For example, the alarm display 323 can indicate critical parameters (e.g., temperature, pH, pressure, etc.) that are deviating from set values. The battery indicator 324 provide visual indication of the unit battery's state of charge (e.g., partially charged, fully charged, etc.). For example, a unit in motion and not connected directly to a power source can instead rely on a charged battery to maintain its function. An alarm can be similarly triggered either locally or via the user interface display 316 when the battery is low, and the unit requires a direct connection to a power source to avoid disruptions to its operation.

The user interface display 316 includes a diagram 325 of the unit being viewed, as selected using the screen navigation tab 317. In the example of FIG. 3, the unit being viewed is a jacketed mixer. The user interface display 316 can include the display of various critical parameters (e.g., temperature, conductivity, pH, etc.) and/or other relevant settings (e.g., pump settings). For example, a first pump 326 and a second pump 328 are connected to the mixer 312 as indicated by the mixer display diagram 325. In some examples, additional information is provided regarding the first and/or second pump(s) 326, 328 (e.g., revolutions per minute (RPM), etc.). Additionally, the user can view and/or adjust unit settings, as shown using an example temperature indicator 332 (e.g., 0.0 degrees Celsius), an example electrical conductivity indicator 334 (e.g., −37.5 millisiemens/centimeter), an example weight indicator 338 (e.g., 312.8 kg), and an example agitation indicator 340 (10.0 RPM). While the example user interface display 316 includes multiple indicators of unit performance and/or status, any other information can be presented and/or displayed to the user via the user interface 315.

FIG. 4 is a block diagram of an example implementation 400 of the bioprocess unit tracker 313 of FIG. 3 to facilitate bioprocess instrument monitoring. The bioprocess unit tracker 313 includes an example locator 402, an example configurator 404, an example settings modifier 406, an example power level identifier 408, an example operating condition identifier 410, an example alert manager 412, and an example database 414.

The locator 402 determines the real-time location of a bioprocess unit (e.g., unit(s) 108, 110, 112, 114, 116 of FIG. 2, and/or unit(s) 306, 312 of FIG. 3). For example, when the bioprocess unit is in motion, the locator 402 determines the unit location based on the level of detail configured during unit set-up (e.g., room, floor, facility, etc.). In some examples, the locator 402 differentiates the unit(s) 108, 110, 112, 114, 116 based on a unique identification (ID) assigned to each of the units (e.g., allowing tracking with the user interface display 316). In some examples, the locator 402 transmits specific coordinates of unit location on the biomanufacturing plant floor, such that the location coordinates can be displayed live to a user via the user interface 315 of FIG. 3. In some examples, the location of multiple units 108, 110, 112, 114, 116 can be used to allocate one or more of the units 108, 110, 112, 114, 116 to specific areas and/or specific processing tasks (e.g., downstream processing/upstream processing, specific batch processing, etc.). For example, the locator 402 of the bioprocess unit tracker 313 determines a location of the mixer 312 and/or the bioreactor 306 of FIG. 3 and the wireless TX/RX 310 in communication with the mixer 312 transmits the data to the controller 104 via the communication interface 206. The controller 104 can track the mixer 312 while the mixer 312 is stationary or in motion, and at the same time determine whether the mixer 312 can be allocated to another section of the processing workflow based on the location and/or status information the controller 104 receives from other operational mixers and/or other bioprocess units (e.g., bioreactor(s) 306, etc.).

The configurator 404 can be used to configure the bioprocess unit(s) 108, 110, 112, 114, 116. For example, the configurator 404 can receive configuration instructions from the controller 104, allowing auto-configuration of the unit when it is connected to the network 210 (e.g., via PnP computing). In some examples, the configurator 404 configures and/or re-configures one or more of the unit(s) 108, 110, 112, 114, 116 to perform specific tasks and/or operations based on anticipated processing needs at particular locations. For example, the unit(s) 108, 110, 112, 114, 116 can be configured to operate in a first mode at a first location (e.g., downstream processing location and/or specific batch processing location) and configured to operate in a second mode at a second location. For example, the bioreactor 306 can be configured by the configurator 404 to operate in a batch, fed-batch, continuous, or perfusion processing mode(s) depending on the location of the bioreactor 306. In some examples, the configurator 404 configures the unit(s) 108, 110, 112, 114, 116 to operate in a first mode (e.g., a batch process mode) or a second mode (e.g., a fed-batch process mode) and/or in a first configuration (e.g., settings specific to a first batch process) or a second configuration (e.g., settings specific to a second batch process). In some examples, the configurator 404 configures the unit(s) 108, 110, 112, 114, 116 to operate according to a first operating condition (e.g., a first set of operating variables such as temperature, pH, gas exchange, etc.) or a second operating condition (e.g., a second set of operating variables) based on the processing mode selected and/or the type of unit configuration performed, as determined based on a type of bioprocessing task to be performed by the unit(s) 108, 110, 112, 114, 116, the bioprocess task being a downstream processing task or an upstream processing task. Additionally, the unit(s) 108, 110, 112, 114, 116 can be configured to process a designated volume of biomass under set environmental conditions. In some examples, the unit(s) 108, 110, 112, 114, 116 can be configured to display and/or transmit an alarm notification when critical parameters are outside a designated range. In some examples, the unit(s) 108, 110, 112, 114, 116 can be configured to operate on battery power when in transit from a first location to a second location. Additionally, the configurator 404 can be used to determine the total processing time (e.g., mixing time, etc.) in which the unit should engage for a specific bioprocessing procedure.

The settings modifier 406 modifies settings for one or more unit(s) 108, 110, 112, 114, 116. For example, initial settings for a specific processing operation (e.g., agitator shaft power, rate of stirring, flow rate, etc.) can be modified based on input from the controller 104 via the communication interface 206. In some examples, the requested change in settings can be initiated via the user interface 315 based on displayed critical parameters (e.g., temperature, conductivity, pH, etc.). The settings modifier 406 can be used to adjust unit settings before and/or during a processing operation. In some examples, the settings can be modified based on unit location and/or anticipated processing task(s) to be performed at the designated unit location, as determined using the controller 104.

The power level identifier 408 identifies the unit 108, 110, 112, 114, 116 battery's state of charge (e.g., partially charged, fully charged, etc.). For example, a unit in motion and not connected directly to a power source can instead rely on a charged battery to maintain its function. In some examples, the power level identifier 408 can trigger an alarm via the user interface 315 when the battery requires charging in advance of anticipated transit from a first location to a second location, thereby allowing the unit to remain operational during transit and maintain the proper environmental conditions necessary for continued processing needs.

The operating condition identifier 410 identifies the operating condition(s) for unit(s) 108, 110, 112, 114, 116 depending on unit location, processing task (e.g., downstream/upstream processing, batch processing, etc.), and/or location of collection points for a specific batch. For example, the controller 104 can determine a change in location of the unit(s) 108, 110, 112, 114, 116, which can affect unit operating conditions. For example, a mixer (e.g., mixer 312) in transit from one batch collection point to another batch collection point may not require adjustments in its operating condition. However, a mixer in transit from a first location requiring upstream processing tasks to a second location requiring a downstream processing task can require a change of its operating conditions. As such, the operating condition identifier 410 determines the operating condition of the unit(s) 108, 110, 112, 114, 116 based on the type of processing task to be completed at a designated location of, and/or time point in, the biomanufacturing process.

The alert manager 412 determines any alerts and/or notifications to trigger locally and/or transmit via the wireless TX/RX 215, 225, 240, 255 of FIG. 2 and/or the wireless TX/RX 304, 310 of FIG. 3 to the controller 104. For example, deviations of critical parameters (e.g., temperature, pH, conductivity, etc.) from a tolerance limit for one or more nominal values can result in an alert to the user via the user interface 315. In some examples, the alert manager 412 can trigger an alert based on low battery power. In some examples, the alert manager 412 identifies settings which lack in consistency over the course of a processing task (e.g., agitator shaft power, rate of stirring, etc.) if the settings are intended to remain consistent over the course of the processing task. In some examples, the alert manager 412 triggers an alert related to manual operations (e.g., bag changes in mixers) that are not performed and/or confirmed prior to commencement of an operation.

The database 414 stores any logged data and/or any other information received from the bioprocess unit during operation (e.g., using local storage, network-based storage, cloud-based storage, etc.). In some examples, the database 414 includes data related to unit location as determined using the locator 402, configurations of the unit as performed using the configurator 404, settings modifications as performed using the settings modifier 406, power level status as determined using the power level identifier 408, operating condition changes as determined using the operating condition identifier 410, and/or generated alerts as determined using the alert manager 412. In some examples, in the event of an unexpected break in communication between the unit(s) 108, 110, 112, 114, 116 and the communication interface 206 and/or the controller 104, the database 414 retains any information (e.g., operating condition, mode, etc.) associated with the unit (e.g., via non-volatile data storage). However, any other information related to the bioprocess unit to which the bioprocess unit tracker 313 is communicatively coupled (e.g., unit tracker 313 coupled to the movable mixer 312 and/or the movable bioreactor 306) can be stored using the database 414. The database 414 can be implemented by any storage device and/or storage disc for storing data such as, for example, flash memory, magnetic media, optical media, etc. Furthermore, the data stored in the database 414 can be in any data format such as, for example, binary data, comma delimited data, tab delimited data, structured query language (SQL) structures, etc. While in the illustrated example the database 414 is illustrated as a single database, the database 414 can be implemented by any number and/or type(s) of databases.

While an example implementation of the bioprocess unit tracker 313 is illustrated in FIG. 4, one or more of the elements, processes and/or devices illustrated in FIG. 4 may be combined, divided, re-arranged, omitted, eliminated and/or implemented in any other way. Further, the example locator 402, the example configurator 404, the example settings modifier 406, the example power level identifier 408, the example operating condition identifier 410, the example alert manager 412, and/or, more generally, the example bioprocess unit tracker 313 of FIG. 4 may be implemented by hardware, software, firmware and/or any combination of hardware, software and/or firmware. Thus, for example, any of the example locator 402, the example configurator 404, the example settings modifier 406, the example power level identifier 408, the example operating condition identifier 410, the example alert manager 412, and/or, more generally, the example bioprocess unit tracker 313 of FIG. 4 can be implemented by one or more analog or digital circuit(s), logic circuits, programmable processor(s), programmable controller(s), graphics processing unit(s) (GPU(s)), digital signal processor(s) (DSP(s)), application specific integrated circuit(s) (ASIC(s)), programmable logic device(s) (PLD(s)) and/or field programmable logic device(s) (FPLD(s)). When reading any of the apparatus or system claims of this patent to cover a purely software and/or firmware implementation, at least one of the example locator 402, the example configurator 404, the example settings modifier 406, the example power level identifier 408, the example operating condition identifier 410, the example alert manager 412, and/or, more generally, the example bioprocess unit tracker 313 of FIG. 4 is/are hereby expressly defined to include a non-transitory computer readable storage device or storage disk such as a memory, a digital versatile disk (DVD), a compact disk (CD), a Blu-ray disk, etc. including the software and/or firmware. Further still, the example bioprocess unit tracker 313 of FIG. 4 may include one or more elements, processes and/or devices in addition to, or instead of, those illustrated in FIG. 4, and/or may include more than one of any or all of the illustrated elements, processes and devices. As used herein, the phrase “in communication,” including variations thereof, encompasses direct communication and/or indirect communication through one or more intermediary components, and does not require direct physical (e.g., wired) communication and/or constant communication, but rather additionally includes selective communication at periodic intervals, scheduled intervals, aperiodic intervals, and/or one-time events.

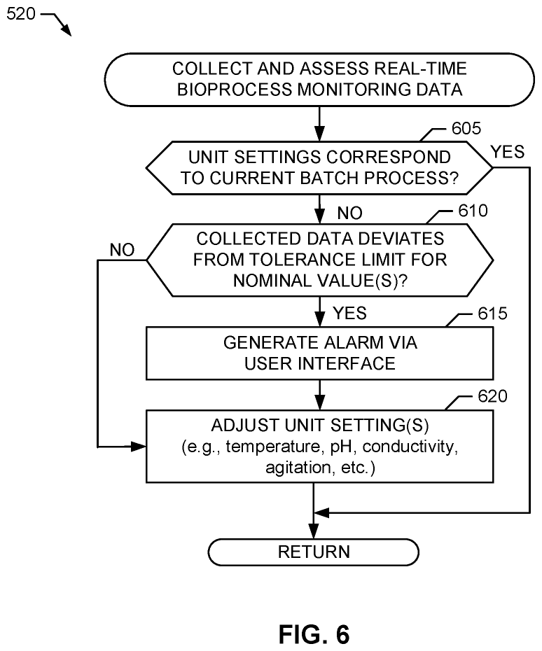

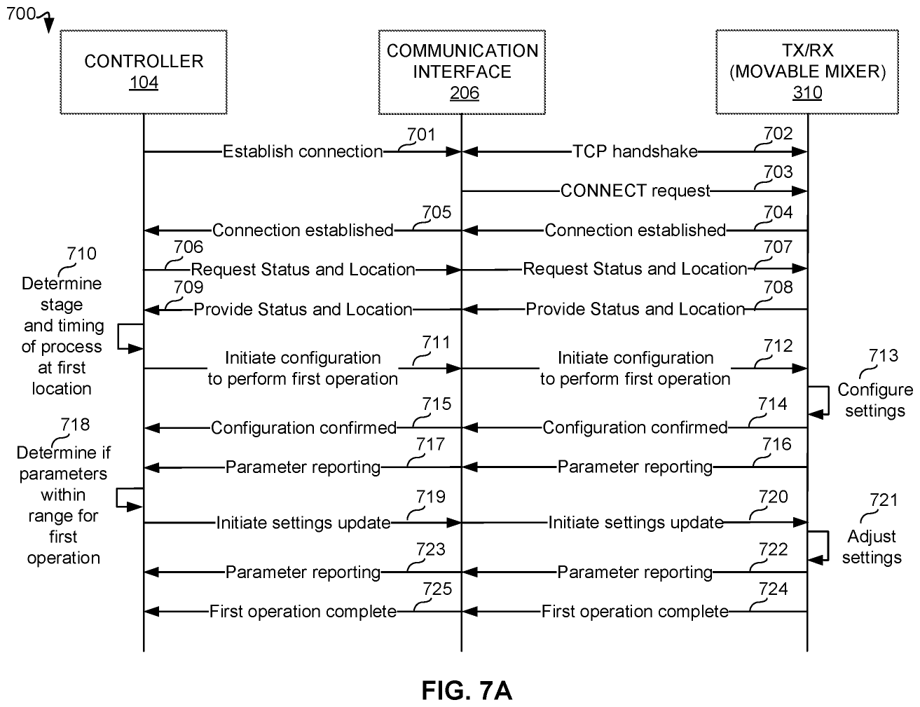

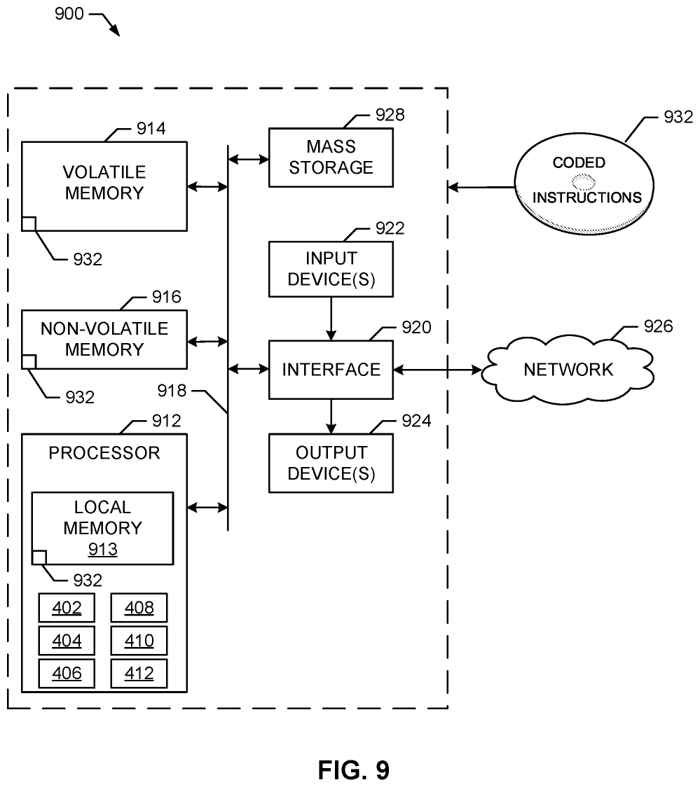

Flowcharts representative of example hardware logic, machine readable instructions, hardware implemented state machines, and/or any combination thereof for implementing the bioprocess unit tracker 313 of FIG. 4 are shown in FIGS. 5-6. The machine readable instructions may be one or more executable programs or portion(s) of an executable program for execution by a computer processor such as the processor 912 shown in the example processor platform 900 discussed below in connection with FIG. 9. The program may be embodied in software stored on a non-transitory computer readable storage medium such as a CD-ROM, a floppy disk, a hard drive, a DVD, a Blu-ray disk, or a memory associated with the processor 912, but the entire program and/or parts thereof could alternatively be executed by a device other than the processor 912 and/or embodied in firmware or dedicated hardware. Further, although the example program is described with reference to the flowchart illustrated in FIGS. 5-6, many other methods of implementing the example bioprocess unit tracker 313 may alternatively be used. For example, the order of execution of the blocks may be changed, and/or some of the blocks described may be changed, eliminated, or combined. Additionally or alternatively, any or all of the blocks may be implemented by one or more hardware circuits (e.g., discrete and/or integrated analog and/or digital circuitry, an FPGA, an ASIC, a comparator, an operational-amplifier (op-amp), a logic circuit, etc.) structured to perform the corresponding operation without executing software or firmware.

The machine readable instructions described herein may be stored in one or more of a compressed format, an encrypted format, a fragmented format, a compiled format, an executable format, a packaged format, etc. Machine readable instructions as described herein may be stored as data (e.g., portions of instructions, code, representations of code, etc.) that may be utilized to create, manufacture, and/or produce machine executable instructions. For example, the machine readable instructions may be fragmented and stored on one or more storage devices and/or computing devices (e.g., servers). The machine readable instructions may require one or more of installation, modification, adaptation, updating, combining, supplementing, configuring, decryption, decompression, unpacking, distribution, reassignment, compilation, etc. in order to make them directly readable, interpretable, and/or executable by a computing device and/or other machine. For example, the machine readable instructions may be stored in multiple parts, which are individually compressed, encrypted, and stored on separate computing devices, wherein the parts when decrypted, decompressed, and combined form a set of executable instructions that implement a program such as that described herein.

In another example, the machine readable instructions may be stored in a state in which they may be read by a computer, but require addition of a library (e.g., a dynamic link library (DLL)), a software development kit (SDK), an application programming interface (API), etc. in order to execute the instructions on a particular computing device or other device. In another example, the machine readable instructions may need to be configured (e.g., settings stored, data input, network addresses recorded, etc.) before the machine readable instructions and/or the corresponding program(s) can be executed in whole or in part. Thus, the disclosed machine readable instructions and/or corresponding program(s) are intended to encompass such machine readable instructions and/or program(s) regardless of the particular format or state of the machine readable instructions and/or program(s) when stored or otherwise at rest or in transit.

The machine readable instructions described herein can be represented by any past, present, or future instruction language, scripting language, programming language, etc. For example, the machine readable instructions may be represented using any of the following languages: C, C++, Java, C#, Perl, Python, JavaScript, HyperText Markup Language (HTML), Structured Query Language (SQL), Swift, Ladder Logic, Function Block Diagram (FBD), Structured Text, Sequential Flow Charts, Instruction List, etc.

As mentioned above, the example processes of FIGS. 5-6 may be implemented using executable instructions (e.g., computer and/or machine readable instructions) stored on a non-transitory computer and/or machine readable medium such as a hard disk drive, a flash memory, a read-only memory, a compact disk, a digital versatile disk, a cache, a random-access memory and/or any other storage device or storage disk in which information is stored for any duration (e.g., for extended time periods, permanently, for brief instances, for temporarily buffering, and/or for caching of the information). As used herein, the term non-transitory computer readable medium is expressly defined to include any type of computer readable storage device and/or storage disk and to exclude propagating signals and to exclude transmission media.

FIG. 5 is a flowchart representative of example machine-readable instructions 500 that may be executed to monitor a bioprocess instrument. As described in connection with FIGS. 2-3, the controller 104 initiates communication with bioprocess unit(s) 108, 110, 112, 114, 116 via the communication interface 206. The bioprocess unit(s) 108, 110, 112, 114, 116 are communicatively coupled to one or more wireless TX/RX (e.g., TX/RX 215, 225, 240, 255), allowing the unit(s) 108, 110, 112, 114, 116 to transmit data to, and/or receive data from, the controller 104. The bioprocess unit tracker 313 of FIG. 4 facilitates the collection and/or retrieval of data from the bioprocess unit(s) 108, 110, 112, 114, 116. For example, the locator 402 determines the location of the unit(s) 108, 110, 112, 114, 116 (block 510). In some examples, the locator 402 identifies the room, area, floor, and/or facility of the bioprocessing plant where the bioprocess unit is located. In some examples, the location determined by the locator 402 is presented to a user via the user interface 315 of FIG. 3 (e.g., using the user interface display 316).

The operating condition identifier 410 determines a mode, a configuration, and/or an operating condition (e.g., a first operating condition) of the unit(s) 108, 110, 112, 114, 116 (block 515). In some examples, the operating condition of the unit(s) 108, 110, 112, 114, 116 is determined based on the unit location (e.g., within an area dedicated to downstream processing or upstream processing tasks). For example, the bioreactor 306 can operate in several modes, including batch, fed-batch, continuous, and/or perfusion processing. The processing mode(s) of the bioreactor 306 can be dependent on its location, with the processing mode including a specific operating condition (e.g., length of operating time, critical parameter settings, etc.). In some examples, the operating condition(s) depend on the type of operation being performed (e.g., extraction, filtration, purification, sterilization, etc.). As such, the operating condition identifier 410 determines the unit's operating condition and/or assigns an operating condition to the unit(s) 108, 110, 112, 114, 116 based on input from the controller 104.

During unit 108, 110, 112, 114, 116 operation, the controller 104 and/or the bioprocess unit tracker 313 collects and assesses real-time, continuous bioprocess monitoring data (block 520). Additionally, the data logger logs data received from on-line, real-time sensors, at-line analyzers, and/or other automatic data acquisition systems associated with the bioprocess units 108, 110, 112, 114, 116. Whether the unit 108, 110, 112, 114, 116 is in motion or stationary, real-time process monitoring data is collected and assessed to determine deviations from set critical parameter ranges and/or other settings associated with the unit's operating condition(s), as described in more detail in connection with FIG. 6. The operating condition identifier 410 can determine whether the unit 108, 110, 112, 114, 116 has completed a given process and/or operation (block 525). In some examples, the unit 108, 110, 112, 114, 116 can be configured to maintain the same operating condition(s) for a designated period of time (e.g., for the duration of downstream processing and/or upstream processing). If the operating condition identifier 410 determines that the process is complete (block 525), the controller 104 and/or bioprocess unit tracker 313 determines whether additional processing is needed (e.g., upstream/downstream, specific batch processing, etc.). If no further processing is needed, the unit 108, 110, 112, 114, 116 can be shut-off and/or placed in standby mode until the controller 104 transmits additional processing requests (e.g., via the wireless TX/RX 215, 225, 240, 255) and/or initiates unit 108, 110, 112, 114, 116 relocation to another area of the biomanufacturing facility (block 560).

If the controller 104 and/or bioprocess unit tracker 313 determines that the process is not yet complete, the locator 402 identifies unit location and determines whether the unit is in motion (block 530). If the unit is not in motion (e.g., in transit from a first location to a second location) the controller 104 and/or the bioprocess unit tracker 313 continues to collect and assess real-time bioprocess monitoring data (block 520). If the locator 402 determines that the unit has moved to the second location, the operating condition identifier 410 determines a second operating condition of the unit 108, 110, 112, 114, 116 at the second location identified by the locator 402 (block 535). In some examples, the controller 104 assigns the unit's operating condition at the second location in real-time. In some examples, the configurator 404 configures the unit to operate using specific operating condition(s) during unit configuration and/or re-configuration. If the operation condition identifier 410 determines that the operating condition at the unit's second location is not different from the unit's operating condition at the first location (block 540), the controller 104 and/or the bioprocess unit tracker 313 continue to collect and assess real-time bioprocess monitoring data (block 520). If the operating condition identifier 410 determines that operating conditions of the unit at the second location are different from the first location, the settings modifier 406 adjusts bioprocess unit 108, 110, 112, 114, 116 settings to match the required operating conditions at the second location (block 545). For example, the settings modifier 406 can modify the unit settings (e.g., temperature, conductivity, agitation, etc.) based on the requirements of the updated operating conditions and/or the type of bioprocess unit being monitored (e.g., bioreactor, mixer, pump, fermenter, etc.).

In some examples, the operating condition identifier 410 determines whether any manual procedures have been completed prior to the unit starting a new operation at the second location (block 550). For example, a bioprocess unit such as the mixer 312 of FIG. 3 can require disposable bag changes prior to the commencement of a new mixing process. In some examples, a user can be prompted via a local display connected to the unit and/or the user interface 315 of FIG. 3 to complete the manual set-up process and/or confirm that the process has been completed (block 555). In some examples, the alert manager 412 issues an alert and/or notification to the user of the unit (e.g., via the user interface 315) to indicate that a manual procedure has not been performed. Once the required manual adjustment(s) have been completed, the operations to be performed are initiated and the controller 104 and/or the bioprocess unit tracker 312 continue to collect and assess real-time monitoring data (block 520) until the process has ended (block 525) and no further processing is required and/or scheduled (block 560).

For example, a bioprocess unit (e.g., the bioreactor 306 and/or the mixer 312 of FIG. 3) can be used in upstream and/or downstream processing. In some examples, the upstream process (cell culture selection, culture media preparation, identification of growth parameters needed to optimize cell growth and biopharmaceutical production, etc.) is dedicated to microbial growth needed to produce biopharmaceuticals and/or other biomolecules (e.g., viral vectors, plasmids, gene therapies, vaccines, monoclonal antibodies, etc.). The bioreactor 306 can be used to establish well-controlled conditions needed to transform substrates into metabolic products during upstream processing. In some examples, the bioreactor 306 can be configured, using the configurator 404, to operate in a first mode (e.g., batch) at a first location (e.g., for a first upstream process or a first stage of the first process) and a second mode (e.g., fed-batch) at a second location (e.g., for a second upstream process or a second stage of the first process). Depending on the designated mode, the configurator 404 can configure the bioreactor 306 to operate according to a first operating condition (e.g., at a set temperature, pH, oxygen supply, etc.). As such, the locator 402 initially determines the location of the bioreactor 306 to identify whether the unit is in an upstream and/or downstream processing area (block 510). For example, downstream processing involves purification of a biological product to yield a final purified product, including initial recovery, purification, and polishing (e.g., removal of contaminants and/or unwanted forms of the target biomolecule). Once the location of the unit 306 is confirmed, the operating condition identifier 410 determines the bioreactor's first operating condition (block 515). For example, the bioreactor 306 can be operating in a batch-process mode according to operating conditions specified by that mode (e.g., temperature, pH, oxygen levels, etc.). In some examples, the bioreactor's operating conditions can change based on changes in the mode and/or a given bioprocessing task (e.g., total cell media volume, etc.), which can directly depend on the bioreactor's location on the biomanufacturing plant floor.

During the bioreactor's operation, the controller 104 and/or the unit tracker 313 collect and assess real-time bioprocess monitoring data (block 520) to determine whether the unit is continuing to maintain optimal environmental conditions (e.g., pH, temperature, gas supply, etc.) for cell expansion. In some examples, the bioreactor 306 can be moved from a first location in upstream processing to a second location in upstream processing, or to a first or a second location in downstream processing. During transit, the controller 104 and/or the unit tracker 313 continue to receive and monitor data relevant to the bioreactor's performance (e.g., settings, critical parameters, etc.). Continuous monitoring ensures thorough documentation of the processing conditions while allowing ongoing control and/or adjustment of settings if any deviations are noted during transit and/or when the unit is stationary. If the bioreactor 306 is moved to a second location during upstream processing, the operating condition identifier 410 determines unit operating conditions at the second location (block 535). In some examples, the configurator 404 can adjust the bioreactor 306 mode from the first mode (e.g., batch) to the second mode (e.g., fed-batch), which can result in updated bioreactor 306 operating conditions at the bioreactor's second location. For example, in batch mode, all nutrients can be supplied in an initial base medium, in fed-batch mode, nutrients can be added once they are depleted, while in perfusion mode medium can be circulated in the bioreactor through a growing culture to allow simultaneous supply of nutrients, product harvesting, and/or waste removal. Therefore, a change in the bioreactor's mode from batch to fed-batch can be achieved by automatic adjustments of bioreactor 306 settings (e.g., using the settings modifier 406 at block 545). In some examples, additional manual adjustments (e.g., bag changes, tube connections, etc.) can be performed to ensure the bioreactor 306 is ready for processing at the second location (block 550). As such, the bioprocess units (e.g., bioreactor 306, mixer 312) can be used at multiple locations and for multiple purposes (e.g., use of the unit for multiple steps within a batch campaign) and re-allocated based on processing needs.

FIG. 6 is a flowchart representative of example machine-readable instructions 520 that may be executed to collect and assess real-time bioprocess monitoring data. In the example of FIG. 6, the controller 104 and/or the settings modifier 406 determine whether unit 108, 110, 112, 114, 116 settings correspond to a current batch process and/or an operating condition (block 605). For example, once the operating condition identifier 410 identifies the operating condition(s) at a second location of the bioprocess unit (e.g., once the unit has been moved to a second location), the settings modifier 406 can be used to modify settings to match the new operating conditions. Additionally, the controller 104 and/or the operating condition identifier 410 of the bioprocess unit tracker 313 determine whether collected monitoring data (e.g., data logged by the data logger 303) deviates from a tolerance limit for nominal value(s) (block 610). For example, temperature fluctuations can be tolerated within a certain range, as determined via unit configuration and/or controller-based monitoring of the logged data. In some examples, the alert manager 412 (and/or the controller 104) generates an alarm via the user interface 315 to indicate data deviations from a set tolerance limit (block 615). Such a response can be necessary to maintain controlled environmental conditions required to maintain high product quality and process reproducibility, while also adhering to regulatory standards. If the collected data is not found to deviate from set tolerance limits (block 610), the settings modifier 406 adjusts unit settings directly to ensure their correspondence to the ongoing process operation(s) (block 620). As such, the bioprocess monitoring data is evaluated continuously and in real-time to ensure conformance to set deviation tolerance limits and maintenance of optimal operating conditions.

As described in connection with FIG. 5, the bioreactor 306 can operate in a batch mode, a fed-batch mode, perfusion, and/or continuous mode. During bioreactor 306 monitoring, the controller 104 and/or the unit tracker 313 can determine whether bioreactor 306 settings correspond to a current batch process (block 605). For example, cell culture can require stringent conditions (e.g., precise control of temperature, agitation, gas supply, pumping for addition and removal of liquids, exhaust with heating or condensation, etc.). As such, the bioreactor 306 can also include integrated units (e.g., a pump, a scale, etc.) (e.g., as shown using units 112, 114 of FIG. 2) that can also be controlled and/or managed using the controller 104 and/or the bioprocess unit tracker 313. For example, a pump can have a set point of ⅖ mL/min, with a run time of 30 minutes each day starting at a designated time (e.g., 8:00 am). Any deviations of critical parameters (e.g., temperature, pH, etc.) (block 610) during processing can result in loss of product, reduced product quality, and/or reduced product yield (block 545). As such, the settings modifier 406 can adjust bioreactor settings to ensure proper operating conditions are maintained. For example, in addition to temperature and pH levels, glucose levels can be targeted to a specific concentration (e.g., 3 g/L), depending on cell consumption rates. Other concentrations (e.g., lactate, ammonia, etc.) can also be monitored to, for example, ensure peak cell densities and subsequent product yields (e.g., antibody titers, etc.).

FIG. 7A depicts an example data flow diagram 700 for managing communication among the controller 104, the communication interface 206, and the transmitter/receiver (TX/RX) 310 of a bioprocess instrument at a first location. The controller 104 initializes a connection 701 with the bioprocess unit 312 (e.g., a movable mixer) via the communication interface 206. For example, the communication interface 206 connects to the wireless TX/RX 310 via a transmission control protocol (TCP) handshake 702 to allow for the exchange of data between the unit 312 and the controller 104. The communication interface 206 proceeds to send a CONNECT request 703 to establish a connection with a remote end-point (e.g., unit 312). Once the connection is successfully established, the wireless TX/RX 310 confirms the connection at 704, which is communication by the communication interface 206 to the controller 104 at 705. In some examples, the controller 104 sends a request 706 to obtain unit status and location information. The request for unit status and location is communicated to the unit wireless TX/RX 310 via the communication interface 206 at 707. In some examples, the location and status of the unit 312 can be determined using the bioprocess unit tracker 313 of FIGS. 3-4 (e.g., via the locator 402 and/or the operating condition identifier 410). The wireless TX/RX 310 can transmit the unit location information (e.g., specific location coordinates, room, floor, area of the biomanufacturing facility, etc.) and/or the unit status (e.g., ongoing operation, on stand-by, unit is shut-off, ongoing configuration, etc.) at 708 to the communication interface 206, which passes the information to the controller 104 at 709.

Using the status and/or location information retrieved by the controller 104 at 709, the controller 104 can determine the stage and/or timing of the bioprocess at the unit's location (e.g., a first location) at 710. For example, a unit located in Room A of the bioprocess facility can be designated for a specific batch process. In some examples, the timing of the upstream and downstream processing steps can dictate the operations to be performed by the unit 312. For example, a bioreactor 306 can operate in a first mode (e.g., batch) at a first location of upstream processing and in a second mode (e.g., fed-batch) at a second location of upstream processing. As such, the controller 104 can send a request 711 to initiate configuration of the unit 312 to perform an operation (e.g., a first operation) according to the unit's location, as well as the timing and/or stage of a particular upstream and/or downstream process at the given moment in time. The request to configure the unit 312 is transmitted to the wireless TX/RX 310 at 712 via the communication interface 206. In some examples, the configurator 404 of FIG. 4 configures and/or re-configures the unit 312 to perform the first operation (e.g., adjusts unit settings, etc.) at 713. For example, the bioreactor 306 can be configured to maintain specific critical parameters (e.g., temperature, pH, oxygen supply, glucose concentration, etc.) to ensure cell expansion during upstream processing at the first location, but these critical parameters and/or settings (e.g., agitation) can require adjustment and/or unit reconfiguration at the second location (e.g., depending on type of cells being cultured, total volume of culture media, etc.). Once configuration is complete, the wireless TX/RX 310 transmits confirmation 714 to the communication interface 206, which is received by the controller 104 at 715.

Once the first operation is initiated at the unit 312, the bioprocess unit tracker 313 continually transmits parameters (e.g., critical parameters) via the TX/RX 310 at 716. The controller 104 performs ongoing, real-time monitoring of process parameters (e.g., temperature, pH, etc.) and operational settings (e.g., agitation, etc.) based on the unit-specific parameter reporting transmitted to the controller 104 at 717. In some examples, the controller 104 determines, at 718, whether the reported parameters (e.g., logged by the data logger 303 of FIG. 3) are within an acceptable range specific to the first operation. Additionally, the continuous and real-time monitoring of units such as a moving mixer or a bioreactor allows for undisturbed agitation cycles to maintain an optimal environment (e.g., for cell culture media). Deviation of the process parameters from established values and/or ranges can trigger the controller 104 to transmit a request 719 to initiate settings updates, which is transmitted via the communication interface 206 to the TX/RX 310 at 720. For example, if settings are timely adjusted, the total cell density can be maintained at levels necessary to produce a desired amount of final product using a batch or fed-batch process. In some examples, monitoring can be performed using in-line sensors, at line sensors, multi-sensor arrays, etc. The bioprocess unit tracker 313 can initiate setting updates via the settings modifier 406 at 721. Ongoing transmission of parameter data continues at 722 via the communication interface 206, allowing continuous unit monitoring and assessment of changes in the controlled environment within the unit 312. As such, the controller 104 obtains real-time data, at 723, related to parameter reporting provided by the TX/RX 310. Once the first operation is complete, the unit 312 transmits confirmation at 724 that the process has concluded. For example, once cell expansion is completed, the resulting biomass can be used in further downstream processing steps to extract and purify the final product (e.g., an antibody). In some conditions, the operating condition identifier 410 determines process completion and initiates the updated status transmission, which is received by the controller 104 at 725.

FIG. 7B depicts an example data flow diagram 750 for managing communication among the controller 104, the communication interface 206, and the transmitter/receiver (TX/RX) 310 of a bioprocess instrument at a second and a third location. Once the first operation is completed by the unit 312 as described in connection with FIG. 7A, the controller 104 can request a status and location update at 751, which is transmitted to the TX/RX 310 at 752 via the communication interface 206. The bioprocess unit tracker 313 can be used to provide an updated status and/or location of the unit 312 (e.g., using the locator 402 and/or the operating condition identifier 410), which is transmitted to the communication interface 206 at 753 and received by the controller 104 at 754. The controller 104 determines the stage and timing of the process at the unit's updated location (e.g., a second location). If the unit's second location is different than the unit's first location, the controller 104 can transmit a communication at 756 to the unit 312 to initiate configuration of the unit to perform a second operation as determined based on the unit's location and/or the stage and timing of the ongoing bioprocess tasks at the second location of the biomanufacturing facility. For example, a bioreactor 306 can be seamlessly transitioned from operating in a first mode (e.g., batch) to operating in a second mode (e.g., fed-batch) when the unit is moved to the second location, thereby resulting in adjusted operating conditions. In some examples, the bioreactor 306 can remain in the same mode (e.g., batch) but adjust from the first operating condition (e.g., requiring higher glucose concentrations) to the second operating condition (e.g., requiring lower glucose concentration) based on the total number of cells being expanded and/or the required final biomass volume. Once the request is received by the TX/RX 310 at 757, the configurator 404 configures and/or re-configures unit settings at 758 to correspond to the second operation to be performed by the unit at the second location. Once the configuration is performed, a confirmation is transmitted to the controller 104 at 759 via the communication interface 206. The controller 104 receives the confirmation of completed unit configuration at 760.

As the second operation is initiated at unit 312, real-time reporting of process parameters occurs over the duration of the second operation at 761. The controller 104 monitors the incoming data to determine, at 763, if the reported parameters at 762 are within a tolerated range for the second operation. Once the second operation is completed, the bioprocess unit 312 transmits a confirmation 764 of completion, which is received by the controller 104 at 765. In some examples, the controller 104 can determine a change in location of the bioprocess unit 312 based on, for example, a type of batch process, a number of bioprocess units available, and/or the location of collection points for a batch process. For example, the number of mixers already operating in a given area of the biomanufacturing facility dedicated to downstream processing can affect whether mixer 312 is assigned to the same area or a different area of the facility. Likewise, an available mixer 312 already configured to perform an operation at the first location that now needs to be performed (e.g., using the same settings, etc.) at the second location can be allocated more quickly than a mixer involved in upstream processing under a different set of operating conditions. In the example of FIG. 7B, the controller 104 assigns unit 312 to a third location (e.g., different from the first and second locations) based on process stage, timing, and/or number of units (e.g., total number of mixers available). The controller 104 can initiate the assignment via communication 766 to the communication interface 206, which initiates a transmission of the unit location assignment at 767. The configurator 404 can configure the unit 312, at 768, for the third operation to be performed at the third location. In some examples, the unit 312 can be moved by a user from the second location to the third location once the unit 312 has been assigned to the third location. Before and/or during transit, the power level identifier 408 can track the battery power of the unit to ensure that the unit will remain functional when in motion and not directly connected to a power source.

Once the unit 312 is configured to perform the third operation, the unit's TX/RX 310 transmits confirmation of the configuration completion at 769, which is received by the controller 104 at 770. The controller 104 continues to monitor unit 312 status and location via communication 771, which is received by the TX/RX 310 at 772. The TX/RX 310 transmits, at 773, unit location and status as identified using the bioprocess unit tracker 313. In some examples, the unit 312 can be moved from one area of the biomanufacturing facility to another area during an ongoing operation (e.g., the third operation). For example, a batch process can involve multiple collection points, allowing a mixer 312 to be moved from one collection point to another collection point while maintaining the same operating condition(s) and/or updating the operating condition(s) based on process requirements (e.g., changes in temperature, agitation, etc.) and/or process stage (e.g., total collection volume, etc.). The controller 104 monitors process parameters continuously and in real-time when the unit 312 is stationary or in transit. In some examples, the unit 312 is moved to the third location and once the unit is stationary, the third operation begins. For example, once the controller 104 receives, at 774, the updated status and/or location of the unit 312, the controller 104 can confirm, at 775, that the unit is at the third location where it is assigned to perform the third operation. Once the third operation commences, the unit 312 continues to report process parameters, at 776, throughout the duration of the operation. As the process parameters are received by the controller 104 at 777 and/or logged by the data logger 303, the controller 104 determines, at 778, whether the reported process parameters are within an acceptable range for the third operation. Any deviations from acceptable process parameter ranges and/or values initiates an alert to the user via the user interface 315.

In the examples of FIG. 7B, alarms and setpoints do not require reconfiguration, as the values are retained during and after unit transit. For example, if an operating condition for the mixer 312 does not change from one location to another, the alarms and setpoints remain the same as in the previous location. Additionally, the controller 104 and/or the bioprocess unit tracker 313 can initiate automatic normalization and/or correction of any destabilized process parameter values and/or process settings to maintain a controlled environment within the unit 310. For example, agitation in units 306, 312 can be adjusted to ensure homogenous mixing of cells, gases, and nutrients throughout the culture vessel to prevent the cells from settling to the bottom of the vessel while maintaining a uniform culture temperature. Likewise, operating conditions can vary based on the type of unit (e.g., mixer) being used (e.g., pitched-blade impeller for shear-sensitive cells, packed-bed basket impellers for anchorage-dependent or suspension-based cell cultures, etc.) and/or its surface-to-volume ratio for cell growth, given that mixing can occur using axial flow and/or radial flow. In some examples, a given unit (e.g., a first mixer) can be used to transfer content (e.g., biomass, liquid, etc.) to another unit (e.g., a second mixer) in the event that the first mixer has no more capacity left (e.g., for filling or drawing down). For example, when the first mixer (e.g., mixer 312) is receiving a transfer from a bioreactor unit (e.g., bioreactor 306) and the first mixer has reached full capacity, the second mixer can communicate, via a wireless TX/RX, that the second mixer is empty and can continue the transfer of content (e.g., biomass, etc.) from the bioreactor 306 to the second mixer. In some examples, if the first mixer is feeding a process and becomes empty, the second mixer (e.g., a full mixer) can be used to continue the feeding process. As such, bioprocess units (e.g., the first mixer, the second mixer) can act as primary or secondary sources (e.g., for filing or drawing down) at one or more locations throughout the bioprocess facility.

Once the third operation is complete, the TX/RX 310 initiates a communication 779 confirming operation completion to the controller 104, at 780. Once all process operations to be performed by the unit 312 are completed, the controller 104 can place the unit on stand-by and/or initiate unit shut-down at 781. The shut-down request can be transmitted, at 782, by the communication interface 206 to the TX/RX 310. Once the unit is shut-down, the TCP connection between the communication interface 206 and the wireless TX/RX 310 can be closed at 783.

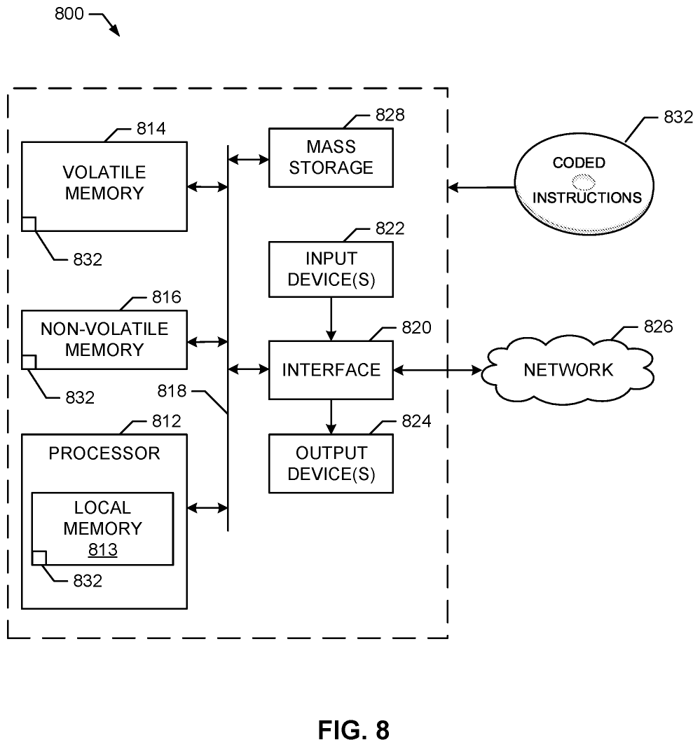

FIG. 8 is a block diagram of an example processing platform 800 structured to execute the example instructions of FIGS. 5-6 to implement the example controller 104 of FIGS. 2 and 3. The processor platform 800 can be a server, a personal computer, a workstation, a self-learning machine (e.g., a neural network), or any other type of computing device.

The processor platform 800 of the illustrated example includes a processor 812. The processor 812 of the illustrated example is hardware. For example, the processor 812 can be implemented by one or more integrated circuits, logic circuits, microprocessors, GPUs, DSPs, programmable logic controllers, or any other controllers from any desired family or manufacturer. The hardware processor may be a semiconductor based (e.g., silicon based) device.

The processor 812 of the illustrated example includes a local memory 813 (e.g., a cache). The processor 812 of the illustrated example is in communication with a main memory including a volatile memory 814 and a non-volatile memory 816 via a bus 818. The volatile memory 814 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 816 may be implemented by flash memory and/or any other desired type of memory device. Access to the main memory 814, 816 is controlled by a memory controller.

The processor platform 800 of the illustrated example also includes an interface circuit 820. The interface circuit 820 may be implemented by any type of interface standard, such as an Ethernet interface, a universal serial bus (USB), a Bluetooth® interface, a near field communication (NFC) interface, and/or a PCI express interface.

In the illustrated example, one or more input devices 822 are connected to the interface circuit 820. The input device(s) 822 permit(s) a user to enter data and/or commands into the processor 812. The input device(s) 822 can be implemented by, for example, an audio sensor, a microphone, a camera (still or video), a keyboard, a button, a mouse, a touchscreen, a track-pad, a trackball, isopoint and/or a voice recognition system.

One or more output devices 824 are also connected to the interface circuit 820 of the illustrated example. The output devices 824 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 820 of the illustrated example, thus, typically includes a graphics driver card, a graphics driver chip and/or a graphics driver processor.

The interface circuit 820 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 826. The communication can be via, for example, an Ethernet connection, a digital subscriber line (DSL) connection, a telephone line connection, a coaxial cable system, a satellite system, a line-of-site wireless system, a cellular telephone system, etc.

The processor platform 800 of the illustrated example also includes one or more mass storage devices 828 for storing software and/or data. Examples of such mass storage devices 828 include floppy disk drives, hard drive disks, compact disk drives, Blu-ray disk drives, redundant array of independent disks (RAID) systems, and digital versatile disk (DVD) drives.

The machine executable instructions 832 of FIGS. 4-5 may be stored in the mass storage device 828, in the volatile memory 814, in the non-volatile memory 816, and/or on a removable non-transitory computer readable storage medium such as a CD or DVD.

FIG. 9 is a block diagram of an example processing platform 900 structured to execute the example instructions of FIGS. 5-6 to implement the example bioprocess unit tracker 313 of FIGS. 3 and 4. The processor platform 900 can be a server, a personal computer, a workstation, a self-learning machine (e.g., a neural network), or any other type of computing device.

The processor platform 900 of the illustrated example includes a processor 912. The processor 912 of the illustrated example is hardware. For example, the processor 912 can be implemented by one or more integrated circuits, logic circuits, microprocessors, GPUs, DSPs, or controllers from any desired family or manufacturer. The hardware processor may be a semiconductor based (e.g., silicon based) device. In this example, the processor 912 implements the example locator 402, an example configurator 404, an example settings modifier 406, an example power level identifier 408, an example operating condition identifier 410, and an example alert manager 412.

The processor 912 of the illustrated example includes a local memory 913 (e.g., a cache). The processor 912 of the illustrated example is in communication with a main memory including a volatile memory 914 and a non-volatile memory 916 via a bus 918. The volatile memory 914 may be implemented by Synchronous Dynamic Random Access Memory (SDRAM), Dynamic Random Access Memory (DRAM), RAMBUS® Dynamic Random Access Memory (RDRAM®) and/or any other type of random access memory device. The non-volatile memory 916 may be implemented by flash memory and/or any other desired type of memory device. Access to the main memory 914, 916 is controlled by a memory controller.

The processor platform 900 of the illustrated example also includes an interface circuit 920. The interface circuit 920 may be implemented by any type of interface standard, such as an Ethernet interface, a universal serial bus (USB), a Bluetooth® interface, a near field communication (NFC) interface, and/or a PCI express interface.

In the illustrated example, one or more input devices 922 are connected to the interface circuit 920. The input device(s) 922 permit(s) a user to enter data and/or commands into the processor 912. The input device(s) 922 can be implemented by, for example, an audio sensor, a microphone, a camera (still or video), a keyboard, a button, a mouse, a touchscreen, a track-pad, a trackball, isopoint and/or a voice recognition system.

One or more output devices 924 are also connected to the interface circuit 920 of the illustrated example. The output devices 924 can be implemented, for example, by display devices (e.g., a light emitting diode (LED), an organic light emitting diode (OLED), a liquid crystal display (LCD), a cathode ray tube display (CRT), an in-place switching (IPS) display, a touchscreen, etc.), a tactile output device, a printer and/or speaker. The interface circuit 920 of the illustrated example, thus, typically includes a graphics driver card, a graphics driver chip and/or a graphics driver processor.

The interface circuit 920 of the illustrated example also includes a communication device such as a transmitter, a receiver, a transceiver, a modem, a residential gateway, a wireless access point, and/or a network interface to facilitate exchange of data with external machines (e.g., computing devices of any kind) via a network 926. The communication can be via, for example, an Ethernet connection, a digital subscriber line (DSL) connection, a telephone line connection, a coaxial cable system, a satellite system, a line-of-site wireless system, a cellular telephone system, etc.

The processor platform 900 of the illustrated example also includes one or more mass storage devices 928 for storing software and/or data. Examples of such mass storage devices 928 include floppy disk drives, hard drive disks, compact disk drives, Blu-ray disk drives, redundant array of independent disks (RAID) systems, and digital versatile disk (DVD) drives.

The machine executable instructions 932 of FIGS. 4-5 may be stored in the mass storage device 928, in the volatile memory 914, in the non-volatile memory 916, and/or on a removable non-transitory computer readable storage medium such as a CD or DVD.

FIGS. 8 and 9 illustrate example processing platforms 800, 900 that can be used to implement the example controller 104 of FIGS. 2 and 3 and the example bioprocess unit tracker 313 of FIGS. 3 and 4. While the example processing platforms 800, 900 are depicted separately, in certain examples, a single processing platform can be used to implement both the example controller 104 of FIGS. 2 and 3 and the example bioprocess unit tracker 313 of FIGS. 3 and 4. However, for purposes of illustration only, the example processing platforms 800, 900 are depicted separately herein.

From the foregoing, it will be appreciated that the above disclosed methods, apparatus, and articles of manufacture improve bioprocess unit tracking and real-time, continuous data monitoring and data logging for a unit in transit between different locations on the biomanufacturing plant floor. Methods and apparatus described herein further permit optimization and improved efficiency of the biomanufacturing process by allowing bioprocess units to be used in upstream processing and/or downstream processing at multiple stages and/or batch collection points, as opposed to remaining stationary as a dedicated unit at a single location, thereby increasing the unit's rate of utilization. Methods and apparatus described herein permit real-time monitoring and continuous data collection during unit transit to ensure tracking of critical parameters (e.g., temperature, pH, conductivity, etc.). In the examples disclosed herein, a primary wireless transmit/receive (TX/RX) interface exchange replaces communication repeater cabinets (e.g., PROFIBUS, CAN, RS-485, etc.), permitting real-time auto-configuration detection between a primary wireless hub and wireless transmitters/receivers in communication with one or more bioprocess units distributed throughout a manufacturing plant. Monitoring of bioprocess units in transit maximizes bioprocess efficiency and improves scalability by allowing units to be used at multiple locations and for multiple purposes (e.g., use of a unit for multiple steps within a batch campaign) and re-allocated based on processing needs, thereby maximizing throughput and total production capacity. For example, a bioprocess unit can be configured and/or reconfigured for use in multiple bioprocess modes (e.g., batch, fed-batch, continuous process, etc.), provide a primary or secondary supply or destination, and/or perform multiple mode-specific operations based on the unit's location and/or the timing and stage of the bioprocess.

Example methods and apparatus for bioprocess monitoring are disclosed herein. Further examples and combinations thereof include the following:

Example 1 includes an apparatus for bioprocess monitoring, the apparatus comprising a controller to monitor a first bioprocess instrument, a data logger to collect data for the first bioprocess instrument, the collected data including data collected while the first bioprocess instrument is transferred from a first location to a second location, a configurator to configure the first bioprocess instrument to operate in a first mode at the first location and in a second mode at the second location, the first or second mode determined based on a type of processing at the first or second location, and a user interface to display the collected data to a user, the collected data including real-time bioprocess monitoring data, the controller to adjust a setting of the first bioprocess instrument based on the monitoring data, the monitoring data used to maintain controlled environmental conditions within a vessel of the instrument during ongoing processing when the instrument is in transit from the first location to the second location.

Example 2 includes the apparatus of example 1, further including a bioprocess unit tracker to manage data collection for the first bioprocess instrument, the unit tracker including a locator to determine a location of the instrument, a settings modifier to modify a setting of the instrument based on the first mode or the second mode, and an operating condition identifier to determine an operating condition of the instrument, the operating condition based on the first mode or the second mode.

Example 3 includes the apparatus of example 1, wherein the collected data includes data collected while the first bioprocess instrument is stationary.

Example 4 includes the apparatus of example 1, wherein the first bioprocess instrument includes a mixer, a bioreactor, a pump, or a scale.

Example 5 includes the apparatus of example 1, wherein the monitoring data includes a temperature, a pH value, or a conductivity.

Example 6 includes the apparatus of example 1, wherein the controller is a programmable logic controller.

Example 7 includes the apparatus of example 1, wherein the first bioprocess instrument is used to transfer intermediate products, buffers, or cell culture media from the first location to the second location.

Example 8 includes the apparatus of example 1, wherein the controller is to generate an alarm notification when the collected data deviates from a tolerance limit for a nominal value.

Example 9 includes the apparatus of example 1, wherein the data logger is to receive the data from the first bioprocess instrument using a wireless transmitter-receiver in communication with the first bioprocess instrument.

Example 10 includes the apparatus of example 1, wherein the first mode includes a first operating condition and the second mode includes a second operating condition, the first and second operating conditions determined based on a type of bioprocess task to be performed by the first bioprocess instrument, the bioprocess task being a downstream processing task or an upstream processing task.

Example 11 includes the apparatus of example 1, wherein the first bioprocess instrument is used for a first batch process, the first and second locations corresponding to locations serving the first batch process.

Example 12 includes the apparatus of example 1, wherein the controller is to monitor the first bioprocess instrument and a second bioprocess instrument, the first instrument used for a first batch process and the second bioprocess instrument used for a second batch process, the controller to determine the change in location of the first or second bioprocess instrument.

Example 13 includes the apparatus of example 12, wherein the controller determines the change in location of the first or second bioprocess instrument based on a type of batch process, a number of bioprocess instruments available, or a location of collection points for the first batch process or the second batch process.

Example 14 includes a method for bioprocess monitoring, the method comprising operating a first bioprocess instrument in a first mode, the first mode based on at least one of a first location or a first bioprocess task to be performed by the first instrument, monitoring the first bioprocess instrument during a transfer of the first bioprocess instrument from the first location to a second location, the monitoring including collection of real-time bioprocess monitoring data, configuring the first bioprocess instrument to operate in a second mode based on at least one of the monitoring data, the second location, or a second bioprocess task, and operating the first bioprocess instrument in the second mode in the second location, a setting of the instrument modified based on the monitoring data.

Example 15 includes the method of example 14, further including managing data collection for the first bioprocess instrument, the managing including determining a location of the instrument, and determining an operating condition of the instrument, the operating condition based on the first mode or the second mode.

Example 16 includes the method of example 14, further including monitoring the first bioprocess instrument and a second bioprocess instrument, the first instrument used for a first batch process and the second bioprocess instrument used for a second batch process.

Example 17 includes the method of example 16, further including determining a change in location of the first or second bioprocess instrument based on a type of batch process, a number of bioprocess instruments available, or a location of collection points for the first batch process or the second batch process.

Example 18 includes a non-transitory computer readable storage medium comprising instructions that, when executed, cause a machine to at least operate a first bioprocess instrument in a first mode, the first mode based on at least one of a first location or a first bioprocess task to be performed by the first instrument, monitor the first bioprocess instrument during a transfer of the first bioprocess instrument from the first location to a second location, the monitoring including collection of real-time bioprocess monitoring data, configure the first bioprocess instrument to operate in a second mode based on at least one of the monitoring data, the second location, or a second bioprocess task, and operate the first bioprocess instrument in the second mode in the second location, a setting of the instrument modified based on the monitoring data.

Example 19 includes the non-transitory computer readable medium of example 18, wherein the instructions, when executed, cause the machine to determine a location of the instrument, and determine an operating condition of the instrument, the operating condition based on the first mode or the second mode.

Example 20 includes the non-transitory computer readable medium of example 18, wherein the instructions, when executed, cause the machine to determine a change in location of the first or second bioprocess instrument based on a type of batch process, a number of bioprocess instruments available, or a location of collection points for a first batch process or a second batch process.

Although certain example methods, apparatus and articles of manufacture have been described herein, the scope of coverage of this patent is not limited thereto. On the contrary, this patent covers all methods, apparatus and articles of manufacture fairly falling within the scope of the claims of this patent.

What is claimed is:

1. An apparatus for bioprocess monitoring, the apparatus comprising:
a controller to monitor a first bioprocess instrument;
a data logger to collect data for the first bioprocess instrument, the collected data including data collected while the first bioprocess instrument is transferred from a first location to a second location,
a configurator to configure the first bioprocess instrument to operate in a first mode at the first location and in a second mode at the second location, the first or second mode determined based on a type of processing at the first or second location; and
a user interface to display the collected data to a user, the collected data including real-time bioprocess monitoring data, the controller to adjust a setting of the first bioprocess instrument based on the monitoring data, the monitoring data used to maintain controlled environmental conditions within a vessel of the instrument during ongoing processing when the instrument is in transit from the first location to the second location.

2. The apparatus of claim 1, further including a bioprocess unit tracker to manage data collection for the first bioprocess instrument, the unit tracker including:
a locator to determine a location of the instrument;
a settings modifier to modify a setting of the instrument based on the first mode or the second mode; and
an operating condition identifier to determine an operating condition of the instrument, the operating condition based on the first mode or the second mode.

3. The apparatus of claim 1, wherein the collected data includes data collected while the first bioprocess instrument is stationary.

4. The apparatus of claim 1, wherein the first bioprocess instrument includes a mixer, a bioreactor, a pump, or a scale.

5. The apparatus of claim 1, wherein the monitoring data includes a temperature, a pH value, or a conductivity.

6. The apparatus of claim 1, wherein the controller is a programmable logic controller.

7. The apparatus of claim 1, wherein the first bioprocess instrument is used to transfer intermediate products, buffers, or cell culture media from the first location to the second location.

8. The apparatus of claim 1, wherein the controller is to generate an alarm notification when the collected data deviates from a tolerance limit for a nominal value.

9. The apparatus of claim 1, wherein the data logger is to receive the data from the first bioprocess instrument using a wireless transmitter-receiver in communication with the first bioprocess instrument.

10. The apparatus of claim 1, wherein the first mode includes a first operating condition and the second mode includes a second operating condition, the first and second operating conditions determined based on a type of bioprocess task to be performed by the first bioprocess instrument, the bioprocess task being a downstream processing task or an upstream processing task.

11. The apparatus of claim 1, wherein the first bioprocess instrument is used for a first batch process, the first and second locations corresponding to locations serving the first batch process.

12. The apparatus of claim 1, wherein the controller is to monitor the first bioprocess instrument and a second bioprocess instrument, the first instrument used for a first batch process and the second bioprocess instrument used for a second batch process, the controller to determine the change in location of the first or second bioprocess instrument.

13. The apparatus of claim 12, wherein the controller determines the change in location of the first or second bioprocess instrument based on a type of batch process, a number of bioprocess instruments available, or a location of collection points for the first batch process or the second batch process.

14. A method for bioprocess monitoring, the method comprising:
operating a first bioprocess instrument in a first mode, the first mode based on at least one of a first location or a first bioprocess task to be performed by the first instrument;
monitoring the first bioprocess instrument during a transfer of the first bioprocess instrument from the first location to a second location, the monitoring including collection of real-time bioprocess monitoring data;
configuring the first bioprocess instrument to operate in a second mode based on at least one of the monitoring data, the second location, or a second bioprocess task; and
operating the first bioprocess instrument in the second mode in the second location, a setting of the instrument modified based on the monitoring data.

15. The method of claim 14, further including managing data collection for the first bioprocess instrument, the managing including:
determining a location of the instrument; and
determining an operating condition of the instrument, the operating condition based on the first mode or the second mode.

16. The method of claim 14, further including monitoring the first bioprocess instrument and a second bioprocess instrument, the first instrument used for a first batch process and the second bioprocess instrument used for a second batch process.

17. The method of claim 16, further including determining a change in location of the first or second bioprocess instrument based on a type of batch process, a number of bioprocess instruments available, or a location of collection points for the first batch process or the second batch process.

18. A non-transitory computer readable storage medium comprising instructions that, when executed, cause a machine to at least:
operate a first bioprocess instrument in a first mode, the first mode based on at least one of a first location or a first bioprocess task to be performed by the first instrument;
monitor the first bioprocess instrument during a transfer of the first bioprocess instrument from the first location to a second location, the monitoring including collection of real-time bioprocess monitoring data;
configure the first bioprocess instrument to operate in a second mode based on at least one of the monitoring data, the second location, or a second bioprocess task; and
operate the first bioprocess instrument in the second mode in the second location, a setting of the instrument modified based on the monitoring data.

19. The non-transitory computer readable medium of claim 18, wherein the instructions, when executed, cause the machine to:
determine a location of the instrument; and
determine an operating condition of the instrument, the operating condition based on the first mode or the second mode.

20. The non-transitory computer readable medium of claim 18, wherein the instructions, when executed, cause the machine to determine a change in location of the first or second bioprocess instrument based on a type of batch process, a number of bioprocess instruments available, or a location of collection points for a first batch process or a second batch process.
